(19) United States
(12) Patent Application Publication
(10) Pub. No.: US 2009/0318451 A1
(43) Pub. Date: Dec. 24, 2009

(54) MORPHOLINE DOPAMINE AGONISTS FOR THE TREATMENT OF PAIN

(75) Inventor: Michael Andrew Ackley, Kent (GB)

Correspondence Address: PFIZER INC;Mary J Hosley, 150 EAST 42ND STREET, MS: 150/02/E112, NEW YORK, NY, 10017-5612, US

(73) Assignee: Pfizer Inc.

(21) Appl. No.: 12/521,596

(22) PCT Filed: Jan. 4, 2008
(86) PCT No.: PCT/IB2008/000041

§ 371 Date: Jun. 29, 2009

Related U.S. Application Data
(60) Provisional application No. 60/891,066, filed on Feb. 22, 2007.

(30) Foreign Application Priority Data
Jan. 15, 2007 (GB) 0700786.7

(51) Int. Cl. A61K 31/5377; A61K 31/535

(52) U.S. Cl. 514/235.5; 514/231.2

(57) ABSTRACT

The present invention relates to use of a compound of formula (I), (Ia), or (Ib):

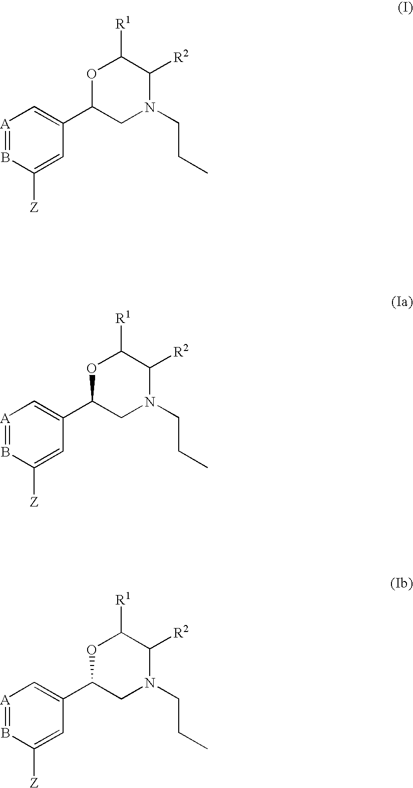

wherein A, B, Z, R¹ and R² have the meanings given in the specification, as a medicament for the treatment of a number of pain conditions, particularly chronic or nociceptive pain, in a mammal.

The present invention relates to a class of dopamine agonists, more particularly a class of agonists that are selective for D3 over D2. These compounds are useful for the treatment and/or prevention of pain, particularly chronic and/or nociceptive pain.

Chronic pain is a common problem affecting around 1 in 5 adults in developed countries. In 30% to 40% of adults, the pain is musculoskeletal and joint in origin, with another 30% being due to neck and back problems. In 1% to 2%, the pain is due to cancer (Bond, et al., “Why pain control matters in a world full of killer diseases” In:Carr D B, ed. Pain Clinical Updates, International Association for the Study of Pain (Information supplier) Online www.iasp-pain.org. September 2004, Vol. 12 No. 4). Chronic pain has a substantial impact on patients' quality of life, and is associated with physical and social disability and psychological distress (McWilliams et al, “Mood and anxiety disorders associated with chronic pain”, Pain, 2003, Vol. 106, No. 1-2, pp. 127-133). Although a variety of analgesic agents are available, including opioids and NSAIDS, many patients remain refractory to these treatments because of inadequate pain relief or intolerable side effects. Thus, there remains a need for the development of additional treatments with the hope that these treatments will be more efficacious or better tolerated.

The D₂ family of dopamine receptors, which consists of D₂, D₃, and D₄, are believed to be involved in the modulation of pain pathways. There is evidence that administration of a nonselective D₂-family agonist can elicit nociception (M. J. Milan, “Descending control of pain”, Prog. Neurobiol., 2002, Vol. 66, pp. 355-474). Other literature suggests that dopamine release in the nucleus accumbens plays an important role in this analgesic effect (Altier, et al., “The role of dopamine in the nucleus accumbens in analgesia”, Life Sci, 1999, Vol. 65, pp. 2269-2287), and it is within the nucleus accumbens that the highest concentrations of D₃ receptors are found.

The present invention provides for a method of treatment of chronic pain or nociceptive pain by administering a compound of formula (I), (Ia) and (Ib):

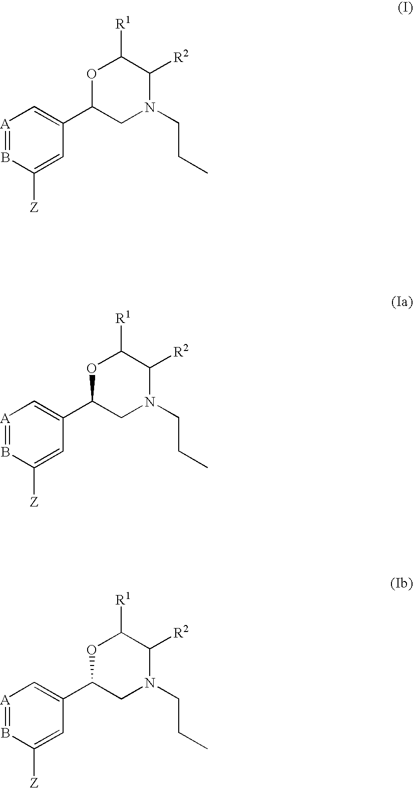

### Wherein:

A is selected from C—X and N,
B is selected from C—Y and N,
R¹ is selected from H and (C₁-C₆)alkyl,
R² is selected from H and (C₁-C₆)alkyl,
X is selected from H, HO, C(O)NH₂, NH₂
Y is selected from H, HO, NH₂, Br, Cl and F
Z is selected from H, HO, F. CONH₂ and CN;
or a pharmaceutically acceptable salt, solvate or prodrug thereof. The pharmaceutically acceptable salts of the compounds of the formula (I) include the acid addition and the base salts thereof.

A pharmaceutically acceptable salt of a compound of the formula (I) may be readily prepared by mixing together solutions of a compound of the formula (I) and the desired acid or base, as appropriate. The salt may precipitate from solution and be collected by filtration or may be recovered by evaporation of the solvent.

Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminum, calcium, magnesium, zinc and diethanolamine salts.

For a review on suitable salts see Berge et al, J. Pharm. Sci., 66, 1-19, 1977.

The pharmaceutically acceptable solvates of the compounds of the formula (I) include the hydrates thereof.

Also included within the present scope of the compounds of the formula (I) are polymorphs thereof.

A compound of the formula (I) contains one or more asymmetric carbon atoms and therefore exists in two or more stereoisomeric forms.

Separation of diastereoisomers may be achieved by conventional techniques, e.g. by fractional crystallisation, chromatography or H.P.L.C. of a stereoisomeric mixture of a compound of the formula (I) or a suitable salt or derivative thereof. An individual enantiomer of a compound of the formula (I) may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

Preferred compounds of the present invention are compounds of formula (Ia) and (Ib).

Particularly preferred are compounds of formula (Ia).

Preferably A is C—X or N and B is C—Y.

More preferably A is N and B is C—Y.

More preferably A is C—X and B is C—Y.

Preferably R¹ is selected from H and (C₁-C₄)alkyl.

More preferably R¹ is H, methyl and ethyl.

Even more preferably R¹ is H or methyl.

Most preferably R¹ is H.

Preferably R² is selected from H and (C₁-C₄)alkyl.

More preferably R² is selected from H, methyl and ethyl.

Most preferably R² is selected from H and methyl.

In a particularly preferred embodiment R² is H.

In a further particularly preferred embodiment R² is methyl.

Preferably X is selected from H, OH and NH₂.

Most preferably X is selected from H and OH.

In a particularly preferred embodiment X is H.

In a further particularly preferred embodiment X is OH.

Preferably Y is selected from H, NH₂, Cl and F.

Most preferably Y is selected from H and NH₂.

In a particularly preferred embodiment Y is H.

In a further particularly preferred embodiment Y is NH₂.

Preferably Z is selected from H, HO and F.

Most preferably Z is selected from H or HO.

In a particularly preferred embodiment Z is H.

In a further particularly preferred embodiment Z is HO.

Particularly preferred are compounds (and salts thereof) of the present invention exemplified herein; more preferred are:
R-(−)-3-(4-Propylmorpholin-2-yl)phenol (Example 7A)
S-(+)-3-(4-Propylmorpholin-2-yl)phenol (Example 7B)
R-(−)-3-(4-Propylmorpholin-2-yl)phenol hydrochloride (Example 8)
R-5-(4-Propylmorpholin-2-yl)benzene-1,3-diol (Example 15A)
S-5-(4-Propylmorpholin-2-yl)benzene-1,3-diol (Example 15B)
R-(+)-2-Fluoro-5-(4-propylmorpholin-2-yl)phenol (Example 23A)
S-(−)-2-Fluoro-5-(4-propylmorpholin-2-yl)phenol (Example 23B)
2-Bromo-4-(4-propylmorpholin-2-yl)phenol (Example 30)
2-Hydroxy-5-(4-propylmorpholin-2-yl)benzamide (Example 35)
2-Nitro-4-(4-propylmorpholin-2-yl)phenol (Example 36)
2-Amino-4-(4-propylmorpholin-2-yl)phenol (Example 37)
5-(4-Propylmorpholin-2-yl)pyridin-2-ylamine (Example 44A and 44B)
2-Chloro-5-(4-propyl-morpholin-2-yl)phenol (Example 54)
3-[(5S)-5-methyl-4-propylmorpholin-2-yl]phenol (Example 60)
5-[(2S,5S)-5-methyl-4-propylmorpholin-2-yl]pyridin-2-amine (Example 66)
5-[(2R,5S)-5-methyl-4-propylmorpholin-2-yl]pyridin-2-amine (Example 67)

Most preferred are:
R-(−)-3-(4-Propylmorpholin-2-yl)phenol (Example 7A)
S-(+)-3-(4-Propylmorpholin-2-yl)phenol (Example 7B)
R-(−)-3-(4-Propylmorpholin-2-yl)phenol hydrochloride (Example 8)
R-5-(4-Propylmorpholin-2-yl)benzene-1,3-diol (Example 15A)
S-5-(4-Propylmorpholin-2-yl)benzene-1,3-diol (Example 15B)
R-(+)-2-Fluoro-5-(4-propylmorpholin-2-yl)phenol (Example 23A)
S-(−)-2-Fluoro-5-(4-propylmorpholin-2-yl)phenol (Example 23B)
5-(4-Propylmorpholin-2-yl)pyridin-2-ylamine (Example 44A and 44B)
2-Chloro-5-(4-propyl-morpholin-2-yl)phenol (Example 54)
5-[(2S,5S)-5-methyl-4-propylmorpholin-2-yl]pyridin-2-amine (Example 66)
5-[(2R,5S)-5-methyl-4-propylmorpholin-2-yl]pyridin-2-amine (Example 67).

In preferred embodiments, the invention comprises:

a method of treating chronic or nociceptive pain in a mammal, comprising administering to said mammal an effective amount of 5-[(2R,5S)-5-methyl-4-propylmorpholin-2-yl]pyridin-2-amine, or a pharmaceutically acceptable salt, solvate or prodrug thereof;

a method of treating chronic pain (preferably chronic nociceptive pain) in a mammal, comprising administering to said mammal an effective amount of a compound of formula (I), (Ia) or (Ib), as defined above, either in its broadest aspect or a preferred aspect, or a pharmaceutically acceptable salt, solvate or prodrug thereof;

a method of treating chronic pain (preferably chronic nociceptive pain) in a mammal, comprising administering to said mammal an effective amount of 5-[(2R,5S)-5-methyl-4-propylmorpholin-2-yl]pyridin-2-amine, or a pharmaceutically acceptable salt, solvate or prodrug thereof;
a method of treating nociceptive pain in a mammal, comprising administering to said mammal an effective amount of a compound of formula (I), (Ia) or (Ib), as defined above, either in its broadest aspect or a preferred aspect, or a pharmaceutically acceptable salt, solvate or prodrug thereof;
a method of treating nociceptive pain in a mammal, comprising administering to said mammal an effective amount of 5-[(2R,5S)-5-methyl-4-propylmorpholin-2-yl]pyridin-2-amine, or a pharmaceutically acceptable salt, solvate or prodrug thereof;
a method of treating pain associated with osteoarthritis in a mammal, comprising administering to said mammal an effective amount of a compound of formula (I), (Ia) or (Ib), as defined above, either in its broadest aspect or a preferred aspect, or a pharmaceutically acceptable salt, solvate or prodrug thereof;
a method of treating pain associated with osteoarthritis in a mammal, comprising administering to said mammal an effective amount of 5-[(2R,5S)-5-methyl-4-propylmorpholin-2-yl]pyridin-2-amine, or a
pharmaceutically acceptable salt, solvate or prodrug thereof;
a method of treating post-surgical pain in a mammal, comprising administering to said mammal an effective amount of a compound of formula (I), (Ia) or (Ib), as defined above, either in its broadest aspect or a preferred aspect, or a pharmaceutically acceptable salt, solvate or prodrug thereof;
a method of treating post-surgical pain in a mammal, comprising administering to said mammal an effective amount of 5-[(2R,5S)-5-methyl-4-propylmorpholin-2-yl]pyridin-2-amine, or a pharmaceutically acceptable salt, solvate or prodrug thereof;
a method of treating neuropathic pain in a mammal, comprising administering to said mammal an effective amount of a compound of formula (I), (Ia) or (Ib), as defined above, either in its broadest aspect or a preferred aspect, or a pharmaceutically acceptable salt, solvate or prodrug thereof;
a method of treating neuropathic pain in a mammal, comprising administering to said mammal an effective amount of 5-[(2R,5S)-5-methyl-4-propylmorpholin-2-yl]pyridin-2-amine, or a pharmaceutically acceptable salt, solvate or prodrug thereof;
a method of treating visceral pain in a mammal, comprising administering to said mammal an effective amount of a compound of formula (I), (Ia) or (Ib), as defined above, either in its broadest aspect or a preferred aspect, or a pharmaceutically acceptable salt, solvate or prodrug thereof.
a method of treating visceral pain in a mammal, comprising administering to said mammal an effective amount of 5-[(2R,5S)-5-methyl-4-propylmorpholin-2-yl]pyridin-2-amine, or a pharmaceutically acceptable salt, solvate or prodrug thereof;
a method of treating inflammatory pain in a mammal, comprising administering to said mammal an effective amount of a compound of formula (I), (Ia) or (Ib), as defined above, either in its broadest aspect or a preferred aspect, or a pharmaceutically acceptable salt, solvate or prodrug thereof; and
a method of treating inflammatory pain in a mammal, comprising administering to said mammal an effective amount of 5-[(2R,5S)-5-methyl-4-propylmorpholin-2-yl]pyridin-2-amine, or a pharmaceutically acceptable salt, solvate or prodrug thereof.

Compounds of the invention may be prepared, in known manner, in a variety of ways. The routes below illustrate methods of synthesising compounds of formula (I): the skilled man will appreciate that compounds of formula (Ia) and (Ib) may be isolated with appropriate resolution techniques.

Compounds of general formula (I) where A is C—X, B is C—Y, R¹ is H or (C₁-C₆)alkyl, R² is H and where X, Y and Z are as described herein may be prepared according to reaction scheme 1.

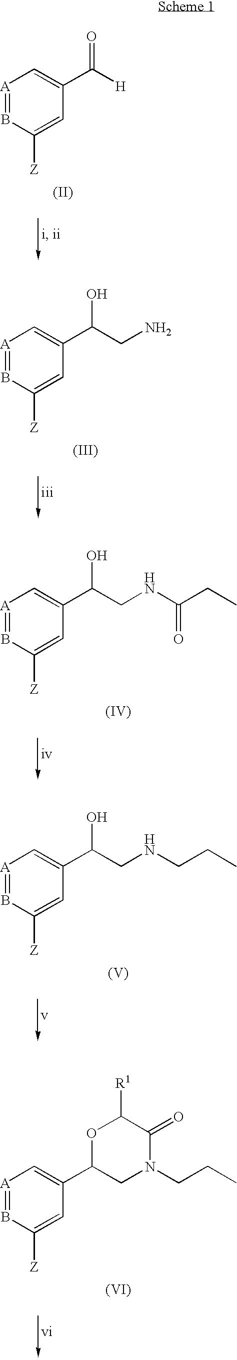

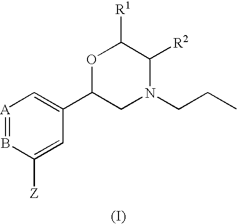

Compounds of formula (III) may be prepared by reacting an aldehyde of formula II with i) a cyanide source or nitromethane followed by ii) reduction with borane, lithium aluminum hydride or hydrogenation. Some compounds of formula II and III are also commercially available.

Compounds of formula (IV) may be prepared by reacting compounds of formula (III) with iii), acid chlorides in the presence of a suitable base such as triethylamine or 4-methylmorpholine. Typical reaction conditions comprise 1.0 equivalents of amine (III), 1.2-2.0 equivalents of base (preferably triethylamine), 1.1-1.3 equivalents of acid chloride in dichloromethane at 25° C.

Compounds of formula (V) may be prepared by reducing compounds of formula (IV) with iv), reducing agents such as borane or lithium aluminum hydride. Typical conditions comprise 1.0 equivalents of amide (IV), 1.2-3.0 equivalents of borane in THF at reflux. Compounds of formula (V) can also be made by reductive animation of compounds of formula (III) with a suitable aldehyde in the presence of sodium cyanoborohydride.

Compounds of formula (VI) may be prepared by reacting compounds of formula V with v), chloroacetyl chloride or 2-substituted chloroacetyl chlorides (such as 2-chloropropionyl chloride or 2-chlorobutyryl chloride) in the presence of base such as triethylamine, sodium carbonate and potassium hydroxide. Typical conditions comprise 1.0 equivalents of amine IV, 1.0-1.3 equivalents of acid chloride, 1.2-2.0 equivalents of triethylamine in dichloromethane at 25° C., the crude reaction mixture is then dissolved in IPA with 1.2-3.0 equivalents of aqueous potassium hydroxide.

Compounds of formula (I) may be prepared by reacting compounds of formula (VI) with vi), reducing agents such as borane or lithium aluminum hydride. Typical conditions comprise 1.0 equivalents of amide VI, 1.2-3.0 equivalents of borane in THF at reflux.

The skilled man will appreciate that due to one of X, Y or Z being a hydroxy group, it will be necessary to protect the hydroxy group(s) with a suitable protecting group throughout the transformations of scheme 1, then remove the protecting group. Methods for deprotection of a phenol group depend on the protecting group. For examples of protection/deprotection methodology see “Protective groups in Organic synthesis”, T W Greene and P G M Wutz. For example, where the hydroxy is protected as a methyl ether, deprotection conditions comprise refluxing in 48% aqueous HBr for 1-24 hours, or by stirring with borane tribromide in dichloromethane for 1-24 hours. Alternatively where the hydroxy is protected as a benzyl ether, deprotection conditions comprise hydrogenation with a palladium catalyst under a hydrogen atmosphere.

Compounds of general formula (I) where one of A or B is N, R¹ is H or (C₁-C₆)alkyl, R² is H and X, Y, and Z are as described herein, with the proviso that one of X, Y or Z is NH₂, may be prepared according to reaction scheme 2. Scheme is illustrated where B is C—Y and where Y is NH₂; the skilled man will understand that the alternative compounds are equally practicable.

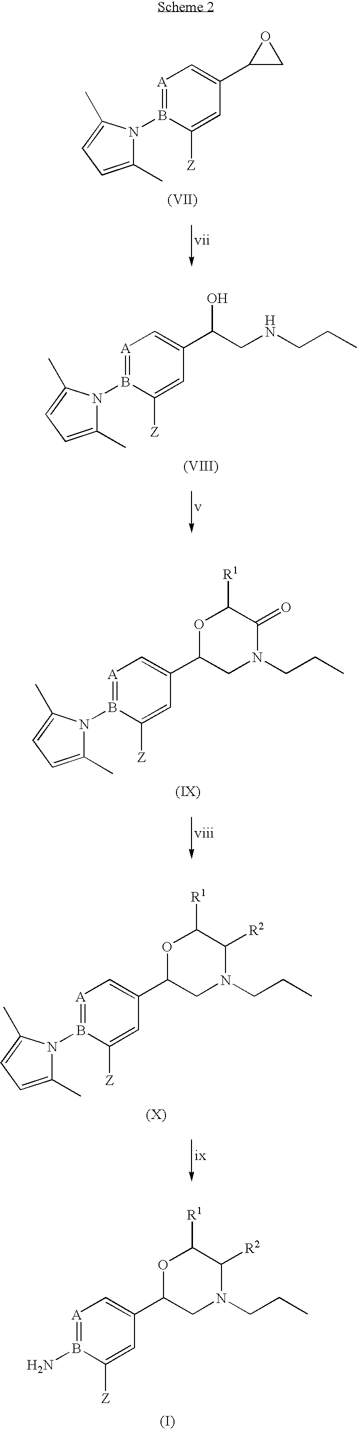

Compounds of formula (VII) may be prepared using the process as described in JP2001048864.

Compounds of formula (VIII) may be prepared by reacting epoxide (VII) with vii), propylamine. Typical reaction conditions comprise stirring the epoxide with excess amine either neat or in dimethylsulphoxide. Compounds of formula (IX) may be prepared by reacting compounds of formula (VIII) with v), chloroacetyl chloride or 2-substituted chloroacetyl chlorides (such as 2-chloropropionyl chloride or 2-chlorobutyryl chloride) in the presence of base such as triethylamine, sodium carbonate and potassium hydroxide. Typical conditions comprise 1.0 equivalents of amine (VIII), 1.2-2.0 equivalents of triethylamine in dichloromethane at 25° C., the crude reaction mixture is then dissolved in IPA with 1.2-3.0 equivalents of aqueous potassium hydroxide.

Compounds of formula (X) may be prepared by reacting compounds of formula (IX) with reducing agents such as lithium aluminum hydride. Typical conditions comprise 1.0 equivalents of amide (X), 1.2 equivalents of lithium aluminum hydride in THF at reflux.

Compounds of formula (I) may be prepared by ix), deprotection. Typical conditions comprise 1.0 equivalents of compound X and 5 equivalents of hydroxylamine hydrochloride in ethanol at reflux.

Compounds of general formula I, where A is C—X, B is C—Y, R¹ is H and R² is H or (C₁-C₆) alkyl and where X, Y and Z are as described herein may be prepared according to reaction scheme 3.

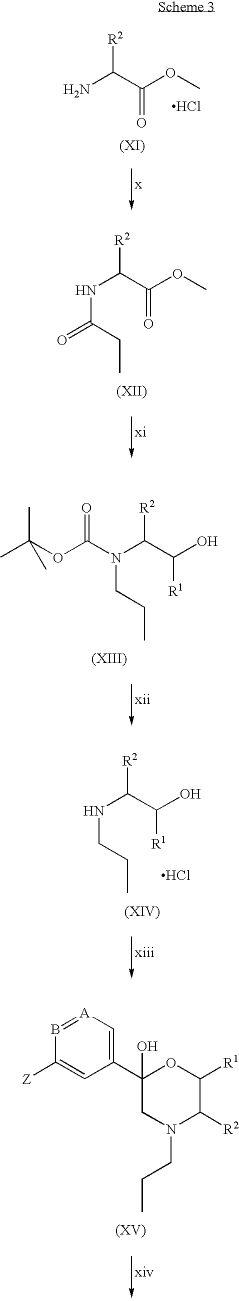

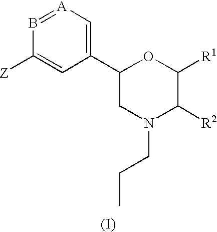

Compounds of the formula (XII) may be prepared by reacting an amino acid ester of the formula (XI) with x) acid chlorides in the presence of a suitable base such as triethylamine and 4-methylmorpholine.

Typical reaction conditions comprise 1 equivalent amino acid ester (XI), 1 equivalent of acid chloride and 3 equivalents of base in dichloromethane at 25° C. Some compounds of formula (XI) are commercially available.

Compounds of the formula (XIII) may be prepared by reacting compounds of the formula (XII) with xi) borane-THF complex, with subsequent breaking of the boron-nitrogen complex with acid and t-butyloxycarbonyl protection of the formed amine. Typical reaction conditions comprise 1 equivalent of the amide (XII) with 3 equivalents of BH₃-THF in THF at reflux, cooling, cautious addition of 6M aqueous HCl, and heating to reflux for a further 6 h. Subsequent evaporation of solvent, redissolution in a methanol:water (8:1) mix, and addition of 5 equivalents of a base such as potassium hydroxide and 1.5 equivalents of di-tert-butyl dicarbonate, and stirring of the mixture for 72 hours.

Compounds of the formula (XIV) may be prepared by reacting compounds of the formula (XIII) with xii) an organic solution of HCl. Typical reaction conditions comprise 1 equivalent of the carbamate (XIII) and a 1-10 equivalents of a 4M solution of HCl in dioxan in dioxan at 25° C.

Compounds of the formula (XV) may be prepared by reacting compounds of the formula (XIV) with xiii) a 2-bromoacetophenone in the presence of a base such as triethylamine or 4-methylmorpholine. The 2-bromoacetophenones may be obtained from commercial sources or alternatively prepared from the parent acetophenone by standard bromination methodology well known to those skilled in the art. Typical conditions comprise 1 equivalent of the aminoalcohol (XIV) with 1-3 equivalents of triethylamine and 1 equivalent of a 2-bromoacetophenone at 65° C.

Compounds of the formula (I) may be prepared by reacting compounds of the formula (XV) with xiv) triethylsilane and trimethylsilyltriflate. Typical conditions comprise addition of 5-10 equivalents of triethylsilane to 1 equivalent of the morpholinol (XV) in dichloromethane at −78° C. followed by addition of 2 equivalents of trimethylsilyltriflate.

The skilled man will appreciate that due to one of X, Y or Z being a hydroxy group, it will be necessary to protect the hydroxy group(s) with a suitable protecting group throughout the transformations of scheme 3, then remove the protecting group. Methods for deprotection of a phenol group depend on the protecting group. For examples of protection/deprotection methodology see “Protective groups in Organic synthesis”, T W Greene and P G M Wutz. For example, where the hydroxy is protected as a methyl ether, deprotection conditions comprise refluxing in 48% aqueous HBr for 1-24 hours, or by stirring with borane tribromide in dichloromethane for 1-24 hours. Alternatively where the hydroxy is protected as a benzyl ether, deprotection conditions comprise hydrogenation with a palladium catalyst under a hydrogen atmosphere.

Compounds of the formula (I) where the stereocentre alpha to the morpholine nitrogen is defined absolutely may be prepared starting from homochiral compounds of the formula (XI), which may be commercially available or obtained through methods readily available to the skilled man in the chemistry literature. The resulting compounds of the formula (I) will contain a mixture of diastereoisomers which may be separated on an HPLC column. Typical conditions comprise eluting through a Chiralcel OJ-H column with 100% MeOH mobile phase.

Compounds of general formula (I) where one of A or B is N, R¹ is H, R² is H or (C₁-C₆)alkyl and X, Y and Z are as described herein, with the proviso that one of X, Y or Z is NH₂, may be prepared according to reaction scheme 4. The scheme is illustrated where B is C—Y and where Y is NH₂; the skilled artisan will understand that the alternative compounds are equally practicable.

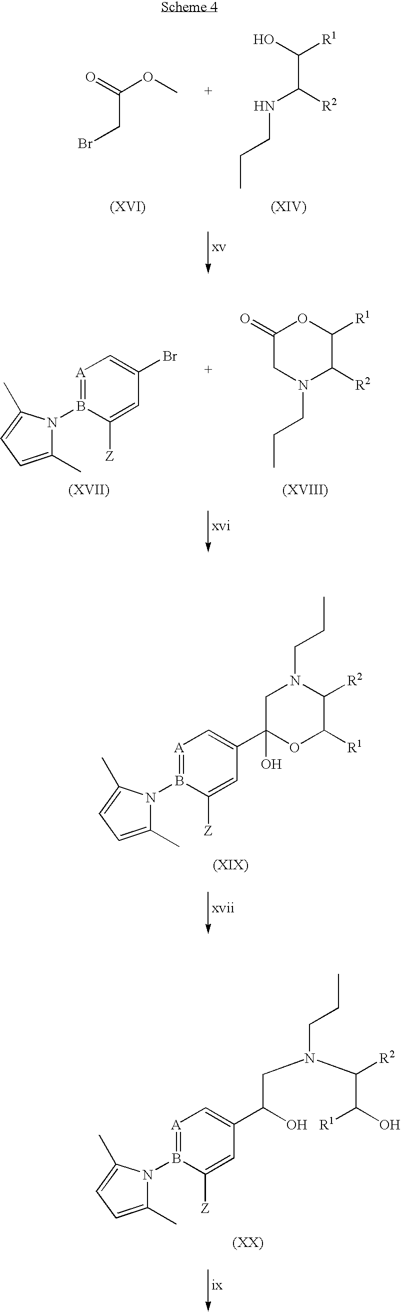

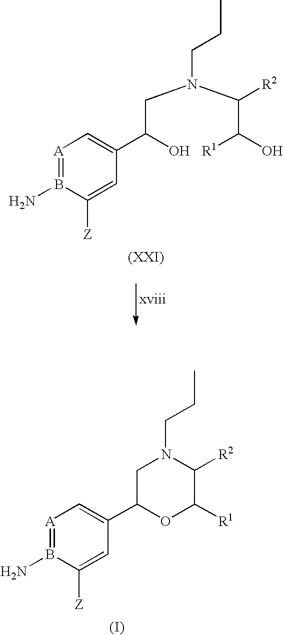

Compounds of formula (XVIII) may be prepared by reacting compounds of formula (XVI) with xv) amino alcohols of formula (XIV) in the presence of a base such as triethylamine or 4-methylmorpholine. Typical conditions comprise 1 equivalent of the aminoalcohol (XIV) with 1-3 equivalents of triethylamine and 1 equivalent of a compound of formula (XVI) using toluene as solvent at room temperature or above. Compounds of formula (XVI) are commercially available.

Compounds of formula (IXX) may be prepared by reacting a compound of formula (XVIII) with xvi) an organometallic reagent formed from the bromide of formula (XVII). Suitable organometallic reagents include Grignard (organomagnesium) or organolithium reagents, which may be prepared from the bromide by halogen metal exchange. Typical conditions comprise addition of isopropylmagensium chloride to the bromide (XVII) in an anhydrous ethereal solvent such as tetrahydrofuran at room temperature (to perform the halogen metal exchange reaction), followed by addition of the morpholinone (XVIII). The bromide (XVII) may be prepared using the process as described in WO9932475.

Morpholinol (IXX) may be reduced to diol (XX) by xvii) reaction with a hydride reducing agent, such as sodium borohydride in an alcohol solvent such as methanol.

Compounds of formula (XXI) may be prepared from the diol (XX) by ix), deprotection. Typical conditions comprise 1.0 equivalents of compound (XX) and 5 equivalents of hydroxylamine hydrochloride in ethanol at reflux.

Compounds of formula (I) may be prepared by xviii) cyclisation of compounds of formula (XXI) by treatment with acid. Typical conditions employ concentrated sulfuric acid and dichloromethane as solvent at room temperature or above.

All of the above reactions and the preparations of novel starting materials using in the preceding methods are conventional and appropriate reagents and reaction conditions for their performance or preparation as well as procedures for isolating the desired products will be well-known to those skilled in the art with reference to literature precedents and the Examples and Preparations hereto.

The compounds of the present invention have utility as selective D3 agonists in the treatment of disease states. There are a number of compounds with activity as both D2 and D3 agonists: however the use of such compounds is associated with a large number of side effects including nausea, emesis, syncope, hypotension and bradycardia, some of which are a cause for serious concern.

It was previously held that the efficacy of the prior art compounds stemmed from their ability to agonize D2; however D2 agonism is implicated as a cause of the side effects detailed above.

The present invention provides a class of selective D3 agonists. Serendipitously, these have been found to be efficacious, whilst reducing the side effects associated with unselective prior art compounds.

Compounds of present invention are useful in treating sexual dysfunction, female sexual dysfunction, including hypoactive sexual desire disorder, sexual arousal disorder, orgasmic disorder and sexual pain disorder; male erectile dysfunction, hypertension, neurodegeneration, psychiatric disorders, depression (e.g. depression in cancer patients, depression in Parkinson's patients, postmyocardial infarction depression, subsyndromal symptomatic depression, depression in infertile women, paediatric depression, major depression, single episode depression, recurrent depression, child abuse induced depression, post partum depression and grumpy old man syndrome), generalized anxiety disorder, phobias (e.g. agoraphobia, social phobia and simple phobias), posttraumatic stress syndrome, avoidant personality disorder, premature ejaculation, eating disorders (e.g. anorexia nervosa and bulimia nervosa), obesity, chemical dependencies (e.g. addictions to alcohol, cocaine, heroin, phenobarbital, nicotine and benzodiazepines), cluster headache, migraine, pain, Alzheimer's disease, obsessive-compulsive disorder, panic disorder, memory disorders (e.g. dementia, amnestic disorders, and age-related cognitive decline (ARCD)), Parkinson's diseases (e.g. dementia in Parkinson's disease, neuroleptic-induced parkinsonism and tardive dyskinesias), endocrine disorders (e.g. hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, attention deficit hyperactivity disorder (ADHD), chronic paroxysmal hemicrania, headache (associated with vascular disorders), emotional lability, pathological crying, sleeping disorder (cataplexy) and shock.

The compounds of formulae (I), (Ia) and (Ib), being selective D3 agonists, are potentially useful in the treatment of a range of disorders. The treatment of pain, particularly chronic and/or nociceptive pain, is a preferred use.

Physiological pain is an important protective mechanism designed to warn of danger from potentially injurious stimuli from the external environment. The system operates through a specific set of primary sensory neurones and is activated by noxious stimuli via peripheral transducing mechanisms (see Millan, 1999, Prog. Neurobiol., 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a hightened sensation of pain. In acute pain these mechanisms can be useful, in promoting protective behaviors which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibres associated with maladaptation and aberrant activity (Woolf & Salter, 2000, Science, 288, 1765-1768). Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 1344). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibres. Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumour related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating. Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp. 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important (Grennan & Jayson, 1994, Textbook of Pain, 397407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 36, 679-686; McCarthy et al., 1994. Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatis and pelvic pain.

It should be noted that some types of pain have multiple aetiologies and thus can be classified in more than one area, e.g. back pain and cancer pain have both nociceptive and neuropathic components.

Other types of pain include:
pain resulting from musculo-skeletal disorders, including myalgia, fibromyalgia, spondylitis, seronegative (non-rheumatoid) arthropathies, non-articular rheumatism, dystrophinopathy, glycogenolysis, polymyositis and pyomyositis;
heart and vascular pain, including pain caused by angina, myocardical infarction, mitral stenosis, pericarditis, Raynaud's phenomenon, scleredoma and skeletal muscle ischemia;
head pain, such as migraine (including migraine with aura and migraine without aura), cluster headache, tension-type headache mixed headache and headache associated with vascular disorders; and
orofacial pain, including dental pain, optic pain, burning mouth syndrome and temporomandibular myofascial pain.

Accordingly, the present invention provides for, the use of a compound of formula (I) in the preparation of a medicament for the treatment or prevention of pain.

Thus, in accordance with a preferred aspect of the invention, there is provided use of a compound of formula (I), (Ia) or (Ib) in the preparation of a medicament for the treatment or prophylaxis of pain, more particularly chronic pain and/or nociceptive pain.

Preferably the compounds of formula (I) are useful in the treatment or prophylaxis of chronic pain and/or nociceptive pain, and most preferably in the treatment or prophylaxis of nociceptive pain.

Preferably said D3 agonist exhibit a functional potency at D3 receptor expressed as an EC50, lower than 100 nM, more preferably lower than 100 nM, yet more preferably lower than 50 nM, most preferably lower than 10 nM.

Preferably said D3 agonist has a selectivity for D3 over D2, wherein said dopamine D3 receptor agonist is at least about 15-timnes, preferably at least about 27-times, more preferably at least about 30-times, most preferably at least about 100-times more functionally selective for a dopamine D3 receptor as compared with a dopamine D2 receptor

Accordingly, the present invention provides for the use of compounds of formula (I), (Ia) or (Ib) in the preparation of a medicament for the treatment of hypertension, premature ejaculation, obesity, cluster headache, migraine, pain, endocrine disorders (e.g. hyperprolactinaemia), vasospasm (particularly in the cerebral vasculature), cerebellar ataxia, gastrointestinal tract disorders (involving changes in motility and secretion), premenstrual syndrome, fibromyalgia syndrome, stress incontinence, trichotillomania and chronic paroxysmal hemicrania, headache (associated with vascular disorders).

### D3/D2 Agonist Bind Assay

Gonazalez et al (Eup. J Pharmacology 272 (1995) R1-R3) discloses an assay for determining the binding capability of a compound at D3 and/or D2 dopamine receptors and thus the binding selectivity of such compounds. This assay is, thus, herein referred to as a binding assay.

### D3/D2 Agonist Functional Assay

A suitable assay for determining functionally the activity of a compound at D3 and/or D2 dopamine receptors is detailed hereinbelow.

Compounds are evaluated as agonists or antagonists at the dopamine D2 and D3 receptors by looking at cAMP levels in a GH4C1 and CHO cell-line expressing the human D2 and D3 receptors, respectively.

### EXPERIMENTAL PROCEDURES

### Inhibition Via Dopamine D3 Receptors of Forskolin-Stimulated Adenylate Cyclase Activity

### Materials

### Cell Culture Media:

hD₃CHO Medium

DMEM, high glucose (Sigma D5671)
2 mM L-Glutamine (Sigma G7513)
10% dialyzed FBS (Sigma F0392)

hD₃CHO (Chinese hamster Ovary) cells expressing the human Dopamine D3 receptor were generated in house. These cells are deficient in the dihydrofolate reductase gene.

Media is made up fresh every week as below, and filtered through a 0.22 μM filter before use. Media is stored at 4° C. and warmed to 37° C. prior to addition to the cells.

### Cell Dissociation Solution (CDS): (Sigma C-5914)

5 ml used to harvest cells from 225 cm² flask (37° C. 5 min for hD2LGH4C1 cells and 10 minutes for hD3CHO cells).

### Phosphate Buffered Saline (PBS): (Gibco. 14040-091)

### Trypan Blue: (Sigma T8154)

### Forskolin (Calbiochem 344273)

Dissolved to a concentration of 20 mM in distilled water. (This stock is stored at +4° C.) 4× assay stock of 40 μM is made by carrying out a 500-fold dilution in PBS buffer. 25 μl of the 40 μM stock is added to a final assay volume of 100 μl, yielding a final assay concentration of 10 μM.

### Test Compounds

Dissolved in 100% DMSO to yield a stock concentration of 10 mM.

### Pramipexole Standard

Dissolved in 100% DMSO to yield a stock concentration of 10 mM.

### Cyclase Activation Flashplate Assay (NEN SMP004B)

Supplied by Perkin-Elmer Life Sciences, Inc

[¹²⁵I]-Cyclic Adenosine Monophosphate (cAMP) (NEX 130)
Supplied by Perkin-Elmer Life Sciences, Inc

### Specific Equipment

### Westbart Microtitre Plate Shaker/Incubator

Packard Topcount NXT (ECADA compatible programme)

### Tecan Genesis

### Labsystems Multi-drop DW

Protocol Testing Compound Activity with hD₃CHO Cells

### Compound Dilutions

Pramipexole is included as a reference standard. A 10-point, semi-log curve is generated every 4 plates. Compound results are normalised to the minimum (0 nM pramipexole) and maximum (100 nM pramipexole) responses generated by the cells. All test compounds may also be tested via a 10-point (semi-log) curve.
Test compounds are dissolved in 100% DMSO to yield a stock concentration of 10 mM. These are further diluted in 100% DMSO to 1 mM via a 10-fold dilution (1000× the final assay concentration required, e.g. 1 mM will give a top concentration of 1 μM).
Pramipexole is dissolved in 100% DMSO to give a concentration of 10 mM. Pramipexole is diluted further to 0.1 mM in 100% DMSO via a 100-fold dilution.
Further dilutions and additions are carried out in 0.4% DMSO/PBS using a suitable Tecan Genesis Protocol, capable of performing serial dilutions at a fold of 3.159 (semi-log unit).

### Tecan Genesis Dilutions

10 μL of the test compounds are added to column 1 of a microplate. 240 μL of 0.4% DMSO/PBS is added to this to give a 25-fold dilution (0.04 mM). 20 μL of the 0.04 mM dilution is transferred to the wells of column 2 where 180 μL of 0.4% DMSO/PBS is added, giving a further 10-fold dilution to achieve a 4×top assay concentration (0.004 mM).
Serial dilutions are performed (3.159-fold) to achieve a semi-log dilution series:
4 μM, 1.27 μM, 400 nM, 127 nM, 40 nM, 13 nM, 4 nM, 1.27 nM, 0.4 nM, 0.1 nM
25 μL (in duplicate) of the serial dilutions are transferred to columns 2-11 of the Flashplate (See Appendix). Since the final assay volume is 100 μL, the final assay concentrations will be:
1000 μM, 317 nM, 100 nM, 32 nM, 10 nM, 3.2 nM, nM, 0.3 nM, 0.1 nM, 0.03 nM
Minimum control (low control): 25 μL 0.4% DMSO/PBS (vehicle) is added to the following wells (column 1 wells E-H and column 2 wells A-D). Cells+forskolin are added later.
Maximum control (high control): 10 mM pramipexole is diluted in PBS via a 250-fold dilution (10 μL+2490 μL PBS) to generate 40 μM pramipexole. 40 μM pramipexole is further diluted via a 100-fold dilution in 0.4% DMSO/PBS (100 μL+9900 μL Vehicle) to generate 400 nM (4× assay concentration of the standard pramipexole). 25 μL of 400 nM pramipexole is added to the following wells of the Flashplate to yield 100 nM pramipexole final; column 1 wells A-D and column 12 wells E-H. Cells+forskolin are added later.

### Cyclase-Activation Flashplate Assay, (NEN SMP004B)

As described in the Materials section, forskolin is dissolved in distilled water to achieve a stock concentration of 20 mM. This is further diluted to 40 μM (4× assay concentration) using PBS. 25 μL of 40 μM stock is added to all wells using a Multi-drop, giving a final concentration of 10 μM. Plates are then sealed and incubated at 37° C. in a Westbart incubator while cells are harvested.
Cells are harvested from flasks, which are between 70%-80% confluent. It is essential that all components added to the cells are warmed to 37° C. 5 mL of CDS is added per T225 flask and incubated at 37° C. for 5 minutes before being neutralised with 5 mL PBS. The cells are then centrifuged at 160 g (1000 rpm) for 5 minutes. The resultant supernatant is discarded and cells are re-suspended in Stimulation Buffer (warmed to 37° C.), to achieve 5×10⁵ cells/ml. 50 μl of cell suspension is then dispensed into all wells of the Flashplate.
Plates are immediately incubated at 37° C. on a shaking incubator for 15 minutes. The reaction is terminated with 100 μl of Detection Mix in all wells (100 μL ¹²⁵I cAMP: 11 ml Detection buffer per plate).
Plates are re-sealed and incubated in the dark for 3 hours to allow equilibrium between the anti-cAMP antibody (coating the wells), [¹²⁵I]-cAMP tracer and cellular cAMP.
Plates are counted on a Packard Topcount NXT using a suitable ECADA compatible protocol (Protocol 75)

### Resuscitation of Frozen Ampoules

Remove ampoules from liquid nitrogen and allow them to equilibrate for 2 minutes as trapped gas or liquid may cause the ampoule to expand rapidly and explode. They can also be placed at minus 20° C. for 2 minutes before thawing.

Thaw ampoules quickly and completely at 37° C. in a water bath.

Transfer cell suspension to a 75 cm² flask containing 10 mL growth media and incubate for 24 h at 37° C., 5% CO₂. After cell attachment (3-6 hours) media is removed and replaced with fresh media (to remove DMSO). After 24 h, if approaching confluency, cells are transferred to a 225 cm² flask. If not, the cells are maintained until they are 70%-80% confluent.

### Cell Harvesting and Splitting

Cells are split on a Friday to provide cells for assays on Monday and Tuesday. Cells required for the remainder of the week are split on a Monday. It is essential not to allow the hD₃CHO cells grow beyond 80% confluence, or to create splits >1:20, as this has detrimental effects on their proliferative response and will subsequently effect the cells ability to perform in the assay.

Cells are grown in 225 cm² flasks (Jumbos). Every component added to the cells must be warmed to 37° C. before use.

### Cell Harvest

Growth media is removed from flasks and cells are washed with warm PBS (Gibco. 14040-091) and removed.
5 mL of cell dissociation buffer is added to cells and placed in incubator for approx. 5 minutes.
Flasks given a sharp tap to dislodge any remaining cells from the tissue culture plastic.
5 mL of PBS is added to the cells and used to wash the base and of the flask. Cells are centrifuged for 5 minutes at 160 g (1000 rpm) to pellet the cells.
Supernatant is discarded and 5 mL of Stimulation Buffer is used to re-suspend the cells. A trypan blue exclusion assay is carried out to determine the number of viable cells.
Cells are diluted in Stimulation Buffer to yield a concentration of 5×10⁵ cells/ml.
To passage to cells the centrifugation step is omitted and the cell suspension is dispensed into new T225 flasks containing 50 mL media.

### Split Ratios

hD₃CHO are split between 1:5 to 1:10. The culture cannot be continued beyond passage 30 as cell line characteristics are lost with increased passage.

### Cryopreservation of Cell Lines

It is essential to create a cell bank of your own cells to resuscitate for further use.
Cells are harvested as described in the previous section. Following the trypan blue exclusion assay, cells are diluted in medium containing 10% DMSO to achieve 2 to 4×10⁵ cells/ml.
Cells are divided into 1 ml aliquots and immediately frozen down gradually, in a ‘Mr Frosty’, (containing fresh IPA) at −80° C. prior to being transferred to a gaseous-phase liquid-nitrogen storage vessel. (Cells may be stored in the ‘Mr Frosty’ for up to 2 days).

It is advisable to test the cell viability by thawing one ampoule after freezing. Viabilities below 70% may cause problems on recovery due to low cell numbers and the presence of debris.

### Data Analysis

The data is analyzed using ECADA.

% Normalisation (in relation to pramipexole) is generated for all compounds via the following formulae:

% Normalisation=(X−B0)/(Max−B0)×100

where,
x=Average net counts for a given concentration of test compound,
Bo=Average net counts of minimum control (0 nM of Pramipexole) and,
Max=Average net counts given of maximum control (100 nM Pramipexole)
Curves can be generated by plotting % normalisation (y) versus concentration of agonist in nM (x). Data is fitted using non-linear regression with the slope constrained to 1. From this, an EC50 and % Emax for the test compound are determined.
Assay Plate Layout (10-point EC50):

| | 1 | 2 | 3 | 4 | 5 | 6 | 7 | 8 | 9 | 10 | 11 | 12 |
|---|---|---|---|---|---|---|---|---|---|---|---|---|
| A | MAX | C1 | | | | | | | | | | MIN |
| B | MAX | C1 | | | | | | | | | | MIN |
| C | MAX | C2 | | | | | | | | | | MIN |
| D | MAX | C2 | | | | | | | | | | MIN |
| E | MIN | C3 | | | | | | | | | | MAX |
| F | MIN | C3 | | | | | | | | | | MAX |
| G | MIN | C4 | | | | | | | | | | MAX |
| H | MIN | C4 | | | | | | | | | | MAX |

Column 1 Wells A-D=MAX: High Controls (cells+forskolin+100 nM pramipexole)
Wells E−H=MIN: Low Controls (cells+forskolin+vehicle)

Column 12: Wells A-D=MIN: Low Controls (cells+forskolin+vehicle)
Wells E−H=MAX: High Controls (cells+forskolin+100 nM pramipexole)

Columns 2-11: 10-point serial dilutions (in duplicate) of test-compounds. Decreasing concentrations from columns 2 to 11 (1000 nM to 0.03 nM). Pramipexole replaces C1 in first plate.

### Inhibition Via Dopamine D2 Receptors of Forskolin Stimulated Adenylate Cyclase Activity

### Materials

### Cell Culture Media:

HD2 GH4C1/hD_{2L} Medium

Hams F-12 (Sigma N6013)
2 mM L-Glutamine (Sigma G7513)
10% FBS (Gibco 10106-169)
700 μg/ml Geneticin (Gibco 10131-019)

GH4C 1/hD_{2L} are rat pituitary cells expressing the human dopamine D2_{long} receptor.
Media is made up fresh every week as below and filtered through a 0.22 μM filter before use. Media is stored at 4° C. and warmed to 37° C. for addition to the cells.

### Cell Dissociation Solution (CDS): (Sigma C-5914)

5 mL used to harvest cells from 225 cm² flask

### Phosphate Buffered Saline (PBS): (Gibco. 14040-091)

### Trypan Blue: (Sigma T8154)

### Forskolin (Calbiochem 344273)

Dissolved to a concentration of 20 mM in distilled water. (This stock is stored at +4° C.).

4× assay stock of 20 μM made by carrying out a 1000-fold dilution in PBS buffer. 25 μl of the 20 μM stock is added to a final assay volume of 100 μl, giving a final assay concentration of 5 μM.

### Test Compounds

Dissolved to a concentration of 10 mM in 100% DMSO

### Pramipexole Standard

Dissolved in 100% DMSO to yield a final stock concentration of 10 mM.

### Cyclase Activation Flashplate Assay (NEN SMP004B)

### Supplied by Perkin-Elmer Life Sciences, Inc

[¹²⁵I]-Cyclic Adenosine Monophosphate (cAMP) (NEX 130)
Supplied by Perkin-Elmer Life Sciences, Inc

### Specific Equipment

### Westbart Microtitre Plate Shaker/Incubator

Packard Topcount NXT (ECADA compatible programme)

### Tecan Genesis

### Labsystems Multi-drop DW

### Protocols

### Compound Dilutions

Pramipexole is included as a reference standard. A 10-point, semi-log curve is generated every 4 plates. Compound responses are normalised to the minimum (0 nM pramipexole) and maximum (1000 nM Pramipexole) responses generated by the cells. All test compounds may also be tested via a 10-point (semi-log) curve.
Test compounds are dissolved in 100% DMSO to yield a stock concentration of 10 mM, (1000× the final assay concentration required, e.g. 10 mM will give a top concentration of 10000 nM).
Pramipexole is dissolved in 100% DMSO to give a concentration of 10 mM. Pramipexole is diluted further to 1 mM in 100% DMSO via a 10-fold dilution.
Further dilutions and additions are carried out in 0.4% DMSO/PBS using a suitable Tecan Genesis Protocol which is capable of performing serial dilutions at a fold of 3.159 (semi-log unit).

### Tecan Genesis Dilutions

10 μL of the test compounds are added to column 1 of a microplate. 240 μL of 0.4% DMSO/PBS is added to this to give a 25-fold dilution (0.4 mM). 20 μL of the 0.4 mM dilution is transferred to wells of column 2 where 180 uL of 0.4% DMSO/PBS is added, giving a further 10-fold dilution to achieve a 4× top assay concentration (0.04 mM).
Serial dilutions are performed (3.159-fold) to achieve a semi-log dilution series:
40 μM, 12.7 μM, 4 μM, 1.27 μM, 400 nM, 130 nM, 40 nM, 13 nM, 4 nM, 1.3 nM 25 μL (in duplicate) of the serial dilutions are transferred to columns 2-11 of the Flashplate (See Appendix). Since the finals assay volume is 100 μL, the final assay concentrations will be:
10,000 μM, 3170 nM, 1000 nM, 320 nM, 100 nM, 32 nM, 1 nM, 3 nM, nM, 0.3 nM

Minimum control (low control): 25 μL of 0.4% DMSO/PBS (vehicle) is added to the following wells (column 1 wells E-H and column 2 wells A-D). Cells and forskolin are added later.
Maximum control (high control): 10 mM pramipexole is diluted in PBS via a 250-fold dilution (10 μL+2490 μL PBS) to generate 40 μM pramipexole. 40 μM pramipexole is further diluted via a 10-fold dilution in 0.4% DMSO/PBS (100 μL+990 μL Vehicle) to generate 4000 nM (4× assay concentration of the standard pramipexole). 25 μL of 40 μM pramipexole is added to the following wells of the Flashplate to yield 1000 nM pramipexole final; column 1 wells A-D and column 12 wells E-H. Cells+forskolin are added later.

### Cyclase-Activation Flashplate Assay, (NEN SMP004B)

As described in the Materials section, forskolin is dissolved in distilled water to achieve a stock concentration of 20 mM. This is further diluted to 20 μM (4× assay concentration) using PBS. 25 μL is added to all wells using a Multi-drop, giving a final concentration of 5 μM. Plates are then sealed and incubated at 37° C. in a Westbart incubator while cells are harvested.
Cells are harvested from flasks which are between 70%-80% confluent. It is essential that all components added to the cells are warmed to 37° C. 5 mL of CDS is added per 225 cm² flask, and incubated at 37° C. for 5 minutes before being neutralised with 5 mL PBS. The cells are then centrifuged at 160 g (1000 rpm) for 5 minutes. The resultant supernatant is discarded and cells are re-suspended in Stimulation Buffer (warmed to 37° C.), to achieve 1×10⁵ cells/ml. 50 μl of cell suspension is then dispensed into all wells of the Flashplate.
Plates are immediately incubated at 37° C. on a shaking incubator for 15 minutes. The reaction is terminated with 100 μl of Detection Mix in all wells (100 μL ¹²⁵I cAMP: 11 ml Detection buffer plate).
Plates are re-sealed and incubated in the dark for 3 hours to allow equilibrium between the anti-cAMP antibody (coating the wells), [¹²⁵I]-cAMP tracer and cellular cAMP.
Plates are counted on a Packard Topcount NXT using a suitable ECADA compatible protocol (Protocol 75)

### Resuscitation of Frozen Ampoules

Remove ampoules from liquid nitrogen and allow them to equilibrate for 2 minutes as trapped gas or liquid may cause the ampoule to expand rapidly and explode. They can also be placed at minus 20° C. for 2 minutes before thawing. Thaw ampoules quickly and completely at 37° C. in a water bath.

Transfer cell suspension to a 75 cm² flask containing 10 mL growth media and incubate for 24 h at 37° C., 5% CO₂. After cell attachment (3-6 hours) media is removed and replaced with fresh media (to remove DMSO). After 24 h, if approaching confluency, cells are transferred to a 225 cm² flask. If not, the cells are maintained until they are 60% confluent.

### Cell Harvesting and Splitting

Cells are split on a Friday to provide cells for assays on Monday and Tuesday. Cells required for the remainder of the week are split on a Monday.

It is essential not to allow the cells grow beyond 60% confluence as this has detrimental effects on their proliferative response and will subsequently effect the cells ability to perform in the assay.

Cells are grown in 225 cm² flasks (Jumbos). Every component added to the cells must be warmed to 37° C. before use.

### Cell Harvest

Growth media is removed from flasks and cells are washed with warm PBS (Gibco. 14040-91) and removed.
5 mL of cell dissociation buffer is added to cells and placed in incubator for approx. 5 minutes.
Flasks given a sharp tap to dislodge any remaining cells from the tissue culture plastic.
5 mL of PBS is added to the cells and used to wash the base and of the flask. Cells are centrifuged for 5 minutes at 160 g (1000 rpm) to pellet the cells.
Supernatant is discarded and 5 mL of Stimulation Buffer is used to re-suspend the cells. A trypan blue exclusion assay is carried out to determine the number of viable cells.
Cells are diluted in Stimulation Buffer to yield a concentration of 1×10⁵ cells/ml.
To passage to cells the centrifugation step is omitted and the cell suspension is dispensed into new T225 flasks containing 50 mL media.

### Split Ratios

GH4C1/D2 are split between 1:3 to 1:5.

### Cryopreservation of Cell Lines

It is essential to create a cell bank of your own cells to resuscitate for further use.
Cells are harvested as described in the previous section. Following the trypan blue exclusion assay, cells are diluted in medium containing 10% DMSO to achieve 2 to 4×10⁰ cells/ml.
Cells are divided into 1 ml aliquots and immediately frozen down gradually, in a ‘Mr Frosty’, (containing fresh IPA) at −80° C. prior to being transferred to a gaseous-phase liquid-nitrogen storage vessel. (Cells may be stored in the ‘Mr Frosty’ for up to 2 days).

It is advisable to test the cell viability by thawing one ampoule after freezing. Viabilities below 70% may cause problems on recovery due to low cell numbers and the presence of debris.

### Data Analysis

The data is analyzed using ECADA.

% Normalisation (in relation to pramipexole) is generated for all compounds via the following formulae:

% Normalisation=(X−B0/(Max−B0)×100

where,
x=Average net counts for a given concentration of test compound,
Bo=Average net counts of minimum control (0 nM of Pramipexole) and,
Max=Average net counts given of maximum control (100 nM Pramipexole)
Curves can be generated by plotting % normalisation (y) versus concentration of agonist in nM (x). Data is fitted using non-linear regression with the slope constrained to 1. From this, an EC50 and % Emax for the test compound are determined.
Assay Plate Layout (10-point EC50):

| | 1 | 2 | 3 | 4 | 5 | 6 | 7 | 8 | 9 | 10 | 11 | 12 |
|---|---|---|---|---|---|---|---|---|---|---|---|---|
| A | MAX | C1 | | | | | | | | | | MIN |
| B | MAX | C1 | | | | | | | | | | MIN |
| C | MAX | C2 | | | | | | | | | | MIN |
| D | MAX | C2 | | | | | | | | | | MIN |
| E | MIN | C3 | | | | | | | | | | MAX |
| F | MIN | C3 | | | | | | | | | | MAX |
| G | MIN | C4 | | | | | | | | | | MAX |
| H | MIN | C4 | | | | | | | | | | MAX |

Column 1: Wells A-D=MAX: High Controls (cells+forskolin+100 nM pramipexole)
Wells E−H=MIN: Low Controls (cells+forskolin+vehicle)
   Column 12: Wells A-D=MIN: Low Controls (cells+forskolin+vehicle)
Wells E−H=MAX: High Controls (cells+forskolin+100 nM pramipexole)

Columns 2-11: 10-point serial dilutions (in duplicate) of test-compounds. Decreasing concentrations from columns 2 to 11 (1000 nM to 0.03 nM). Pramipexole replaces C1 in first plate.

Using the assay described above, the compounds of the present invention all exhibit a functional potency at D3 receptor expressed as an EC50, lower than 1000 nM and a 10 fold selectivity for D3 over D2.

Compound of example 8 has a functional potency at D3 receptor expressed as an EC50, of 7.6 nM and 1315.8 fold selectivity for D3 over D2. Selectivity is calculated as the D2 EC50 value divided by the D3 EC50 value. Where the value of the D2 EC50 was >10000, a figure of 10000 was used in the calculation.

It is to be appreciated that all references herein to treatment include curative, palliative and prophylactic treatment.

Suitable auxiliary active agents for use in the combinations of the present invention include:
1) Naturally occurring or synthetic prostaglandins or esters thereof. Suitable prostaglandins for use herein include compounds such as alprostadil, prostaglandin E₁, prostaglandin E₀, 13,14-dihydroprosta glandin E₁, prostaglandin E₂, eprostinol, natural synthetic and semi-synthetic prostaglandins and derivatives thereof including those described in WO-00033825 and/or U.S. Pat. No. 6,037,346 issued on 14 Mar. 2000 all incorporated herein by reference, PGE₀, PGE₁, PGA₁, PGB₁, PGF₁α, 19-hydroxy PGA₁, 19-hydroxy-PGB₁, PGE₂, PGB₂, 19-hydroxy-PGA₂, 19-hydroxy-PGB₂, PGE₃α, carboprost tromethamine dinoprost, tromethamine, dinoprostone, lipo prost, gemeprost, metenoprost, sulprostune, tiaprost and moxisylate;
2) α—adrenergic receptor antagonist compounds also known as α-adrenoceptors or α-receptors or α-blockers. Suitable compounds for use herein include: the α-adrenergic receptor blockers as described in PCT application WO99/30697 published on 14 Jun. 1998, the disclosures of which relating to α-adrenergic receptors are incorporated herein by reference and include, selective α₁-adrenoceptor or α-adrenoceptor blockers and non-selective adrenoceptor blockers, suitable α₁-adrenoceptor blockers include: phentolamine, phentolamine mesylate, trazodone, alfuzosin, indoramin, naftopidil, tamsulosin, dapiprazole, phenoxybenzamine, idazoxan, efaraxan, yohimbine, rauwolfa alkaloids, Recordati 15/2739, SNAP 1069, SNAP 5089, RS17053, SL 89.0591, doxazocin, terazosin, abanoquil and prazosin; α₂-blocker blockers from U.S. Pat. No. 6,037,346 [14 Mar. 2000] dibenamine, tolazoline, trimazosin and dibenamine; α-adrenergic receptors as described in U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptor blockers include: clonidine, papaverine, papaverine hydrochloride, optionally in the presence of a cariotonic agent such as pirxamine;
3) NO-donor (NO-agonist) compounds. Suitable NO-donor compounds for use herein include organic nitrates, such as mono-di or tri-nitrates or organic nitrate esters including glycerol trinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso-N-acetyl N-acetyl penicillamine (SNAP) S-nitroso-N-glutathione (SNO-GLU), N-hydroxy-L-arginine, amylnitrate, linsidomine, linsidomine chlorohydrate, (SIN-1) S-nitroso-N-cysteine, diazenium diolates, (NONOates), 1,5-pentanedinitrate, L-arginene, ginseng, zizphi fructus, molsidomine, Re-2047, nitrosylated maxisylyte derivatives such as NMI-678-11 and NMI-937 as described in published PCT application WO 0012075;
4) Potassium channel openers or modulators. Suitable potassium channel openers/modulators for use herein include nicorandil, cromokalim, levcromakalim, lemakalim, pinacidil, cliazoxide, minoxidil, charybdotoxin, glyburide, 4-amini pyridine, BaCl₂;
5) Vasodilator agents. Suitable vasodilator agents for use herein include nimodipine, pinacidil, cyclandelate, isoxsuprine, chloropromazine, halo peridol, Rec 1512739, trazodone;
6) Thromboxane A2 agonists;
7) CNS active agents:
8) Ergot alkaloids; Suitable ergot alkaloids are described in U.S. Pat. No. 6,037,346 issued on 14 Mar. 2000 and include acetergamine, brazergoline, bromerguride, cianergoline, delergotrile, disulergine, ergonovine maleate, ergotamine tartrate, etisulergine, lergotrile, lysergide, mesulergine, metergoline, metergotamine, nicergoline, pergolide, propisergide, proterguride and terguride;
9) Compounds which modulate the action of naturetic factors in particular atrial naturetic factor (also known as atrial naturetic peptide), B type and C type naturetic factors such as inhibitors or neutral endopeptidase;
10) Compounds which inhibit angiotensin-converting enzyme such as enapril, and combined inhibitors of angiotensin-converting enzyme and neutral endopeptidase such as omapatrilat.
11) Angiotensin receptor antagonists such as losartan;
12) Substrates for NO-synthase, such as L-arginine;
13) Calcium channel blockers such as amlodipine;
14) Antagonists of endothelin receptors and inhibitors or endothelin-converting enzyme;
15) Cholesterol lowering agents such as statins (e.g. atorvastatin/Lipitor-trade mark) and fibrates;
16) Antiplatelet and antithrombotic agents, e.g. tPA, uPA, warfarin, hirudin and other thrombin inhibitors, heparin, thromboplastin activating factor inhibitors:
17) Insulin sensitising agents such as rezulin and hypoglycemic agents such as glipizide;
18) L-DOPA or carbidopa;
19) Acetylcholinesterase inhibitors such as donezipil;
20) Steroidal or non-steroidal anti-inflammatory agents;

21) Estrogen receptor modulators and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof the preparation of which is detailed in WO 96/21656;
22) A PDE inhibitor, more particularly a PDE 2, 3, 4, 5, 7 or 8 inhibitor, preferably PDE2 or PDE5 inhibitor and most preferably a PDE5 inhibitor (see hereinafter), said inhibitors preferably having an IC50 against the respective enzyme of less than 100 nM (with the proviso that PDE 3 and 4 inhibitors are only administered topically or by injection to the penis):
23) Vasoactive intestinal protein (VIP), VIP mimetic, VIP analogue, more particularly mediated by one or more of the VIP receptor subtypes VPAC1, VPAC or PACAP (pituitary adenylate cyclase activating peptide), one or more of a VIP receptor agonist or a VIP analogue (e.g. Ro-125-1553) or a VIP fragment, one or more of a (L-adrenoceptor antagonist with VIP combination (e.g. Invicorp, Aviptadil);
24) A melanocortin receptor agonist or modulator or melanocortin enhance, such as melanotan II, PT-14, PT-141 or compounds claimed in WO-09964002, WO-00074679, WO-09955679, WO-00105401, WO-00058361, WO00114879, WO-00113112, WO-09954358:
25) A serotonin receptor agonist, antagonist or modulator, more particularly agonists, antagonists or modulators for 5HT1A (including VML 670), 5HT2A, 5HT2C, 5HT3 and/or 5HT6 receptors, including those described in WO-09902159, WO-00002550 and/or WO-00028993;
26) A testosterone replacement agent (including dehydroandrostendione), testosterone (Tostrelle), dihydrotestosterone or a testosterone implant;
27) Estrogen, estrogen and medroxyprogesterone or medroxyprogesterone acetate (MPA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone);
28) A modulator of transporters for noradrenaline, dopamine and/or serotonin, such as bupropion, GW-320659;
29) A purinergic receptor agonist and/or modulator;
30) A neurokinin (NK) receptor antagonist, including those described in WO-09964008;
31) An opioid receptor agonist, antagonist or modulator, preferably agonists for the ORL-1 receptor;
32) An agonist or modulator for oxytocin/vasopressin receptors, preferably a selective oxytocin agonist or modulator;
33) Modulators of cannabinoid receptors;
34) A SEP inhibitor (SEPi), for instance a SEPi having an IC₅₀ at less than 100 nanomolar, more preferably, at less than 50 nanomolar.

Preferably, the SEP inhibitors according to the present invention have greater than 30-fold, more preferably greater than 50-fold selectivity for SEP over neutral endopeptidase NEP EC 3.4.24.11 and angiotensin converting enzyme (ACE). Preferably the SEPi also has a greater than 100-fold selectivity over endothelin converting enzyme (ECE).

By cross reference herein to compounds contained in patents and patent applications which can be used in accordance with invention, we mean the therapeutically active compounds as defined in the claims (in particular of claim 1) and the specific examples (all of which is incorporated herein by reference).

The selective D3 agonists of formulae (I), (Ia) and (Ib) of the present invention may be usefully combined with another pharmacologically active compound, or with two or more other pharmacologically active compounds, particularly in the treatment of pain. For example, a selective D3 agonist, particularly a compound of formula (I), (Ia) or (Ib), or a pharmaceutically acceptable salt or solvate thereof, as defined above, may be administered simultaneously, sequentially or separately in combination with one or more agents selected from:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butalbital, mephobarbital, metharbital, methohexital, pentobarbital, phenobarbital, secobarbital, taibutal, theamylal or thiopental,
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorocyclizine;
a sedative such as glutethimide, meprobamate, methaqualone or dichloralphenazone,

a skeletal muscle relaxant, e.g. baclofen, carisoprodol, chlorzoxazone, cyclobenzaprine, methocarbamol or orphrenadine;

an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (Morphioex®, a combination formulation of morphine and dextromethorphan), topiramate, neramexane or perzinfotel including an NR2B antagonist, e.g. ifenprodil, traxoprodil or (−)-(R)-6-(2-[4-(3-fluorophenyl)-4-hydroxy-1 piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl)quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g. oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesondazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
an alpha-2-delta ligand such as gabapentin, pregabalin, 3-methylgabapentin, (1α,3α,5α)(3-amino-methyl-bicyclo[3.2.0]hept-3-yl)-acetic acid, (3S,5R)-3-aminomethyl-5-methyl-heptanoic acid, (3S,5R>3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-cetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyloctanoic acid;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,I-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazapine, oxaprotiline, fezolamine, tomoxetine, mianserin, bupropion, bupropion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite 0 desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)butyl]thio]-5-chloro-3-pyridinecarbonitrile; 2-4-[(1R,3S)-3-amino-4-hydroxy-1-(5-thiazolyl)butyl]thiol-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N—[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl)amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl),1,4-benzoquinone (CV-6504);
a sodium channel blocker, such as lidocaine;
a 5-HT3 antagonist, such as ondansetron;
   and the pharmaceutically acceptable salts and solvates thereof.

If a combination of active agents is administered, then they may be administered simultaneously, separately or sequentially.

### Auxiliary Agents—PDE5 Inhibitors

The suitability of any particular cGMP PDE5 inhibitor can be readily determined by evaluation of its potency and selectivity using literature methods followed by evaluation of its toxicity, absorption, metabolism, pharmacokinetics, etc in accordance with standard pharmaceutical practice. IC50 values for the cGMP PDE5 inhibitors may be determined using the PDE5 assay (see hereinbelow). Preferably the cGMP PDE5 inhibitors used in the pharmaceutical combinations according to the present invention are selective for the PDE5 enzyme. Preferably (when used orally) they are selective over PDE3, more preferably over PDE3 and PDE4. Preferably (when oral), the cGMP PDE5 inhibitors of the invention have a selectivity ratio greater than 100 more preferably greater than 300, over PDE3 and more preferably over PDE3 and PDE4.

Selectivity ratios may readily be determined by the skilled person. IC50 values for the PDE3 and PDE4 enzyme may be determined using established literature methodology, see S A Ballard et al., Journal of Urology, 1998, vol. 159, pages 2164-2171 and as detailed herein after.

Suitable cGMP PDE5 inhibitors for the use according to the present invention include:
the pyrazolo[4,3-d]pyrimidin-7-ones disclosed in EP-A-0463756; the pyrazolo [4,3-d]pyrimidin-7-ones disclosed in EP-A-0526004; the pyrazolo[4,3-d]pyrimidin-7-ones disclosed in published international patent application WO 93/06104; the isomeric pyrazolo[3,4-d]pyrimidin-4-ones disclosed in published international patent application WO 93/07149; the quinazolin-4-ones disclosed in published international patent application WO 93/12095; the pyrido[3,2-d]pyrimidin-4-ones disclosed in published international patent application WO 94/05661; the purin-6-ones disclosed in published international patent application WO 94/00453, the pyrazolo[4,3-d]pyrimidin-7-ones disclosed in published international patent application WO 98/49166; the pyrazolo[4,3-d]pyrimidin-7-ones disclosed in published international patent application WO 99/54333; the pyrazolo[4,3-d]pyrimidin-4-ones disclosed in EP-A-0995751; the pyrazolo[4,3-d]pyrimidin-7-ones disclosed in published international patent application WO 00/24745; the pyrazolo[4,3-d]pyrimidin-4-ones disclosed in EP-A-0995750; the compounds disclosed in published international application WO95/19978; the compounds disclosed in published international application WO 99/24433 and the compounds disclosed in published international application WO 93/07124. The pyrazolo[4,3-d]pyrimidin-7-ones disclosed in published international application WO 01/27112; the pyrazolo[4,3-d]pyrimidin-7-ones disclosed in published international application WO 01/27113; the compounds disclosed in EP-A-1092718 and the compounds disclosed in EP-A-1092719.

Further suitable PDE5 inhibitors for the use according to the present invention include:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333); (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl)oxy)pyridin-3-yl}-2-methyl-2,6-dihydro-7H-pyrazolo[4,3-d]pyrimidin-7-one (see WO99/54333); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-H-one, also known as 1-(6-ethoxy-5-[3-ethyl-6,7-dihydro-2-(2-methoxyethyl)-7-oxo-2H-pyrazolo[4,3-d]pyrimidin-5-yl]-3-pyridylsulphonyl)-4-ethylpiperazine (see WO 01/27113, Example 8); 5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15); 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66); 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124); 5-(5-Acetyl-2-butoxy-3-pyridinyl)-3-ethyl-2-(1 ethyl-3-azetidinyl)-2,6-dihydro-7H-pyrazolo[4,3-d]pyrimidin-7-one (see WO 01/27112, Example 132); (6R,12aR)-2,3,6,7,12,12a-hexahydro-2-methyl-6-(3,4-methylenedioxyphenyl)-pyrazino[2′, 1′:6,1]pyrido[3,4-]indole-1,4-dione (IC-351), i.e. the compound of examples 78 and 95 of published international application WO95/19978, as well as the compound of examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; and the compound of example 11 of published international application WO93/07124 (EISAI); and compounds 3 and 14 from Rotella D P, J. Med. Chem., 2000, 43, 1257.

Still other suitable PDE5 inhibitors include:
4-bromo-5-(pyridylmethylamino)-6-3-(4-chlorophenyl)-propoxy]-3(2H)pyridazinone; 1-[4-[(1,3-benzodioxol-5-ylmethyl)amino]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocycdopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3 pyridylmethylamino)-6-(3-(4-chlorophenyl) propoxy)-3-(2H)pyridazinone; I-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

The compounds of the formula (I) can be administered alone but will generally be administered in admixture with a suitable pharmaceutical excipient, diluent or carrier selected with regard to the intended route of administration and standard pharmaceutical practice.

Accordingly the present invention provides for us of composition comprising a compound of formula (I), (Ia) or (Ib) and a pharmaceutically acceptable diluent or carrier for the treatment of chronic pain and/or nociceptive pain.

For example, the compounds of the formula (I), (Ia) or (Ib) can be administered orally, buccally or sublingually in the form of tablets, capsules, ovules, elixirs, solutions or suspensions, which may contain flavouring or colouring agents, for immediate-, delayed-, modified-, sustained-, pulsed- or controlled-release applications.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the formula (I), (Ia) or (Ib) may be combined with various sweetening or flavouring agents, colouring matter or dyes, with emulsifying and/or suspending agents and with diluents such as water, ethanol, propylene glycol and glycerin, and combinations thereof.

The compounds of the formula (I), (Ia) or (Ib) can also be administered parenterally, for example, intravenously, intra-arterially, intraperitoneally, intrathecally, intraventricularly, intraurethrally, intrasternally, intracranially, intramuscularly or subcutaneously, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The compounds of formula (I), (Ia) or (Ib) can also be administered intranasally or by inhalation, typically in the form of a dry powder (either alone, as a mixture, for example, in a dry blend with lactose, or as a mixed component particle, for example, mixed with phospholipids) from a dry powder inhaler or as an aerosol spray from a pressurised container, pump, spray, atomizer (preferably an atomizer using electrohydrodynamics to produce a fine mist), or nebuliser, with or without the use of a suitable propellant, such as dichlorofluoromethane.

The pressurised container, pump, spray, atomizer, or nebuliser contains a solution or suspension of the active compound comprising, for example, ethanol (optionally, aqueous ethanol) or a suitable alternative agent for dispersing, solubilising, or extending release of the active, the propellant(s) as solvent and an optional surfactant, such as sorbitan trioleate or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

A suitable solution formulation for use in an atomizer using electrohydrodynamics to produce a fine mist may contain from 1 μg to 10 mg of the compound of the invention per actuation and the actuation volume may vary from 1 μl to 100 μl. A typical formulation may comprise a compound of formula (I), (Ia) or (Ib), propylene glycol, sterile water, ethanol and sodium chloride. Alternative solvents which may be used instead of propylene glycol include glycerol and polyethylene glycol.

Capsules, blisters and cartridges (made, for example, from gelatin or HPMC) for use in an inhaler or insufflator may be formulated to contain a powder mix of the compound of the invention, a suitable powder base such as lactose or starch and a performance modifier such as l-leucine, mannitol, or magnesium stearate.

Formulations for inhaled/intranasal administration may be formulated to be immediate and/or modified release.

Alternatively, the compounds of the formula (I), (Ia) or (Ib) can be administered in the form of a suppository or pessary, or they may be applied topically in the form of a gel, hydrogel, lotion, solution, cream, ointment or dusting powder. The compounds of the formula (I), (Ia) or (Ib) may also be dermally or transdermally administered, for example, by the use of a skin patch. They may also be administered by the pulmonary or rectal routes.

They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the formula (I), (Ia) or (Ib) can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds of the formula (I), (Ia) or (Ib) may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

The present invention is further exemplified by the following, non-limiting examples:

The invention is illustrated by the following non-limiting examples in which the following abbreviations and definitions are used:
α_{D} optical rotation at 587 nm.
Arbocel® filter agent
b broad
Boc tert-butoxycarbonyl
CDCl₃ chloroform-d1
CD₃OD methanol-d4
δ chemical shift
d doublet
dd double doublet
DCM dichloromethane
DMF N,N-dimethylformamide
DMSO dimethylsulfoxide
h hours
HCl hydrogen chloride
LRMS low resolution mass spectrum
m multiplet
m/z mass spectrum peak
min minutes
Mpt melting point
NaOH sodium hydroxide
NMR nuclear magnetic resonance
q quartet
s singlet
t triplet
Tf trifluoroethanesulfonyl
TFA trifluoroacetic acid
THF tetrahydrofuran
TLC thin layer chromatography

Melting points were determined using a Perkin Elmer DSC7 at a heating rate of 20° C./minute).

X-RAY DIFFRACTION DATA WERE RECORDED AT ROOM TEMPERATURE USING A BRUKER AXS SMART-APEX CCD AREA-DETECTOR DIFFRACTOMETER (MO Kα RADIATION). INTENSITIES WERE INTEGRATED FROM SEVERAL SERIES OF EXPOSURES. EACH EXPOSURE COVERED 0.3° IN ω, WITH AN EXPOSURE TIME OF 60 S AND THE TOTAL DATA SET WAS MORE THAN A SPHERE.

### Example 1

### 2-Amino-1-(3-methoxyphenyl)ethanol

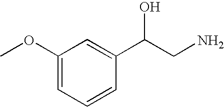

3-Methoxybenzaldehyde (27.2 g, 0.2 mol) in THF (150 ml) was added to a stirred solution of 3N HCl (aq) (150 ml, 0.3 mol) and sodium sulphite (37.8 g. 0.3 mol) at room temperature. After 10 minutes potassium cyanide (19.53 g, 0.3 mol) was added, portion wise, and the reaction mixture was then stirred for 30 minutes. Diethyl ether (800 ml) and water (300 ml) were added and subsequent layers partitioned. Aqueous re-extracted with diethyl ether (500 ml) the organics combined, dried over anhydrous magnesium sulphate, filtered then concentrated in vacuo to give the cyanohydrin intermediate as a colourless oil, (35.57 g. 0.22 mol, >100%). Borane-tetrahydrofuran complex (1M in THF) (400 ml, 0.4 mol) was then cautiously added to the cyanohydrin in THF (100 ml). Once effervescence had ceased, stirring was continued at reflux for 1.5 hours under an atmosphere of nitrogen. The reaction mixture was cooled then quenched with methanol (40 ml) before concentrating in vacuo to give a colourless oil. 6M HCl (aq) (200 ml) was added and reaction stirred at reflux for two hours before concentrating in vacuo to give a white solid. This was pre-absorbed onto silica then purified by column chromatography eluting with dichloromethane: methanol: ammonia (90:10:1) to give the title compound as a colourless oil (31.3 g, 0.19 mol, 94%). ¹H NMR (CDCl₃, 400 MHz) δ: 1.60 (bs, 2H), 2.80 (dd, 1H), 3.02 (dd, 1H), 3.46 (s, 1H), 3.81 (s, 3H), 4.60 (dd, 1H), 6.81 (d, 1H), 6.91 (d, 1H), 6.93 (s, 1H), 7.22 (t, 1H). LRMS: m/z 168 (M-H⁺). Analysis found C, 56.66; H, 8.28; N, 6.91%. C₉H₁₃NO₂.1.33 H₂O requires C, 56.33; H, 8.27; N, 7.30%.

### Example 2

### N-[2-Hydroxy-2-(3-methoxyphenyl)ethyl]propionamide

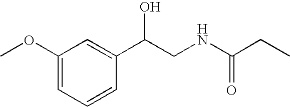

Triethylamine (52 ml, 0.37 mol) was added to the amine from example 1 (31.3 g, 0.19 mol) in dichloromethane (400 ml) and reaction mixture stirred under an atmosphere of nitrogen gas at 0° C. for 10 minutes. Propionyl chloride (16.3 ml, 0.19 mol) was added and after stirring for 30 minutes, the reaction temperature was raised to room temperature for a further 5 hours. The reaction mixture was quenched 1N HCl (aq) (100 ml) and then extracted with dichloromethane (2×50 ml). The organic fractions were combined, dried over anhydrous magnesium sulphate, filtered and concentrated in vacuo to give the title compound as a colourless oil that crystallised on standing to white crystals (28 g, 0.13 mol, 67%). ¹H NMR (CDCl₃, 400 MHz) δ: 1.18 (t, 3H), 2.22 (q, 2H), 2.51 (bs, 1H), 3.31 (m, 1H), 3.71 (dd, 1H), 3.80 (s, 3H), 4.81 (m, 1H), 5.95 (bs, 1H), 6.80 (d, 1H), 6.90 (d, 1H), 6.91 (s, 1H), 7.22 (t, 1H). LRMS: m/z 224. Mpt; 77-78° C. Analysis found C, 63.86; H, 7.82; N, 6.28%. C₁₂H₁₇NO₃.0.1H₂O requires C, 64.04; H, 7.70; N, 6.22%.

### Example 3

### 1-(3-Methoxyphenyl)-2-propylaminoethanol

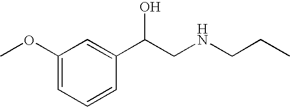

Borane-tetrahydrofuran complex (1M in THF) (376 ml, 0.4 mol) was added to amide from example 2 (28 g, 0.13 mol) in dry THF (100 ml) then the reaction mixture, stirred under an atmosphere of nitrogen gas, was brought to reflux for 2.5 hours. The reaction mixture was cooled then quenched with methanol (40 ml), before concentrating in vacuo to give an opaque white oil. 6N HCl (aq) (200 ml) was added and reaction stirred at reflux for two hours. The reaction mixture was cooled then dichloromethane (200 ml) added and the layers separated. The aqueous layer was rendered basic by addition of potassium carbonate then re-extracted with dichloromethane (2×200 ml). Organic extracts were combined, dried over anhydrous magnesium sulphate, filtered and concentrated in vacuo to give the title compound as a colourless oil that crystallised on standing to give colourless crystals (15.3 g, 0.07 mol, 59%). ¹H NMR (CDCl₃, 400 MHz) δ: 0.93 (t, 3H), 1.62 (q, 2H), 2.71 (q, 2H), 2.81 (t, 2H), 3.00 (d, 1H), 3.80 (s, 3H), 4.30 (bs, 1H), 4.89 (d, 1H), 6.81 (d, 1H), 6.91 (d, 1H), 6.93 (s, 1H), 7.22 (t, 1H). LRMS: m/z 210. Mpt: 50-51° C. Analysis found C, 67.47; H, 9.02; N, 6.45%. C₁₂H₁₉NO₂.0.2H₂O requires C, 67.70; H, 9.19; N, 6.58%.

### Example 4

### 2-Chloro-N-[2-hydroxy-2-(3-methoxyphenyl)ethyl]-N-propylacetamide

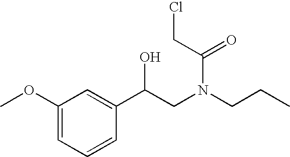

Sodium hydroxide (15.1 g, 0.38 mol) in water (180 ml) was added to the amine from example 3 (15.8 g, 0.08 mol) in dichloromethane (500 ml) and the solution vigorously stirred at room temperature. Chloroacetylchloride (7.22 ml, 0.09 mol) was then added and the reaction mixture stirred for a further 30 minutes. The layers were separated and the aqueous layer re-extracted with dichloromethane (200 ml). The organic extracts were combined, dried over anhydrous magnesium sulphate, filtered and concentrated in vacuo to give the title compound as a colourless oil (17.8 g, 0.06 mol, 83%). ¹H NMR (CDCl₃, 400 MHz) δ: 0.96 (t, 3H), 1.62 (q, 2H), 3.21 (q, 2H), 3.57-3.71 (m, 2H), 3.82 (s, 3H), 4.01-4.21 (bq, 1H), 4.16 (s, 2H), 5.00 (m, 1H), 6.82 (m, 1H), 6.91-6.99 (m, 2H), 7.22 (m, 1H). LRMS: m/z 286. Analysis found C, 57.38; H, 6.95; N, 4.67%. C₁₄H₂₀NO₃Cl.0.33H₂O requires C, 57.64; H, 7.14; N, 4.80%.

### Example 5

### 6-(3-Methoxyyhenyl)-4-propylmorpholin-3-one

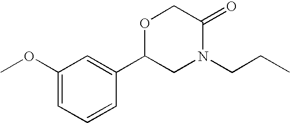

Potassium hydroxide (4.2 g, 0.07 mol), isopropyl alcohol (500 ml) and the amide from example 4 (17.8 g, 0.06 mol) were stirred together as an opaque solution with water (15 ml) for 2 hours. The reaction mixture was concentrated in vacuo and the yellow residue dissolved in ethyl acetate (200 ml). This was partitioned with water (200 ml) then brine (200 ml). The organic fraction was dried over anhydrous magnesium sulphate, filtered and concentrated in vacuo to give the title compound as a yellow oil (15.8 g, 0.06 mol, 100%). ¹H NMR (CDCl₃, 400 MHz) δ: 0.96 (t, 3H), 1.62 (m, 2H), 3.36 (m, 2H), 3.51 (q, 2H), 3.81 (s, 3H), 4.30-4.62 (bq, 2H), 4.79 (d, 1H), 6.85 (d, 1H), 6.91 (d, 1H), 6.95 (s, 1H), 7.29 (t, 1H). LRMS: m/z 272. Analysis found C, 66.80; H, 7.78; N, 5.52%. C₁₄H₁₉NO₃.0.1H₂O requires C, 66.96; H, 7.71; N, 5.58%.

### Example 6

### 2-(3-Methoxyphenyl A4-propylmorpholine

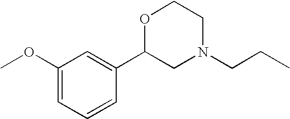

Borane-tetrahydrofuran complex (1M in THF) (200 ml, 0.19 mol) was added dropwise to the morpholin-3-one from example 5 (15.8 g, 0.06 mol) in dry THF (100 ml) under an atmosphere of nitrogen, over 30 minutes. The reaction mixture was brought to reflux for 3 hours then cooled and quenched by addition of methanol (30 ml). The reaction mixture was then concentrated in vacuo and the colourless residue cautiously suspended in 4N HCl (aq) (400 ml) before refluxing for 2.5 hours. The reaction mixture was cooled and dichloromethane (200 ml) added. Layers were separated and the aqueous layer rendered basic by addition of potassium carbonate before re-extracting with dichloromethane (3×100 ml). The organic extracts were combined, dried over anhydrous magnesium sulphate, filtered and concentrated in vacuo to give the title compound as a colourless oil (12.51 g, 0.05 mol, 84%). ¹H NMR (CDCl₃, 400 MHz) δ: 0.95 (t, 3H), 1.59 (q, 2H), 2.05 (t, 1H), 2.23 (t, 1H), 2.40 (t, 2H), 2.81 (d, 1H), 2.98 (d, 1H), 3.80 (5, 3H), 3.85 (t, 1H), 4.05 (d, 1H), 4.60 (d, 1H), 6.81 (d, 1H), 6.91 (d, 1H), 7.21 (t, 1H), 7.23 (s, 1H). LRMS: m/z 236. Analysis found C, 68.94; H, 8.80; N, 5.79%. C₁₄H₂₁NO₂.0.5H₂O requires C, 68.82; H, 9.08; N, 5.73%.

### Example 7A

### R-(−)-3-(4-Propylmorpholin-2-yl)phenol

### Example 7B

### S-(+)-3-(4-Propylmorpholin-2-yl)phenol

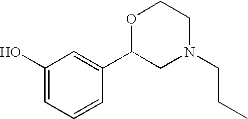

Hydrobromic acid (250 ml) and the anisole from example 6 (8.62 g, 0.03 mol) were heated to reflux together for 1 hour. After cooling the reaction mixture was diluted with water (100 ml) then neutralised by addition of NH₄OH (20 ml). The yellow opaque solution was then extracted with dichloromethane (2×100 ml). The organic extracts were combined then dried over anhydrous magnesium sulphate, filtered and concentrated in vacuo to give the racemic mixture of the title compound as a yellow oil (7.78 g, 0.03 mol, 96%). The enantiomers were separated by chiral chromatography (Chiralpak AD 250*20 mm column) eluting with hexane: isopropyl alcohol: diethylamine (70: 30: 0.05) to give enantiomer 1 (ee>99.5%) and enantiomer 2 (ee>99%). Each enantiomer was purified by column chromatography on silica eluting with dichloromethane:methanol (95:5) to give enantiomer 1(7a) (3.02 g, 0.014 mol, 39%) and enantiomer 2 (7b) (3.15 g, 0.014 mol, 40%) as colourless oils. Enantiomer 1 (7a): ¹H NMR (CDCl₃, 400 MHz) δ: 0.96 (t, 3H), 1.60 (q, 2H), 2.13 (t, 1H), 2.31 (t, 1H), 2.41 (t, 2H), 2.85 (d, 1H), 3.02 (d, 1H), 3.90 (t, 1H), 4.02 (dd, 1H), 4.60 (d, 1H), 6.78 (d, 1H), 6.80 (s, 1H), 6.91 (d, 1H), 7.20 (t, 1H). LRMS: m/z 222 (M-H⁺). Enantiomer 2 (7b): ¹H NMR (CDCl₃, 400 MHz) δ: 0.96 (t, 3H), 1.60 (q, 2H), 2.13 (t, 1H), 2.31 (t, 1H), 2.41 (t, 2H), 2.85 (d, 1H), 3.02 (d, 1H), 3.90 (t, 1H), 4.02 (dd, 1H), 4.60 (d, 1H), 6.78 (d, 1H), 6.80 (s, 1H), 6.91 (d, 1H), 7.20 (t, 1H). LRMS: m/z 222 (M-H⁺).

### Example 8

### R-(−)-3-(4-Propylmorpholin-2-yl)phenol hydrochloride

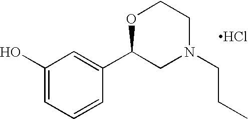

Enantiomer 1 (7a) of example 7 (3.00 g, 0.014 mol) was dissolved in diethyl ether (180 ml) and hydrogen chloride (2.0M solution in diethyl ether) (10 ml) was added. The reaction mixture was stirred at room temperature for 30 minutes, then the solvent was decanted and dried in vacuo, giving title compound as a white solid (3.115 g, 0.012 mol, 90%). ¹H NMR (CD₃OD, 400 MHz) δ: 1.06 (t, 3H), 1.81 (m, 2H), 3.02 (t, 1H), 3.16 (t, 2H), 3.20 (t, 1H), 3.60 (t, 2H), 4.01 (t, 1H), 4.26 (d, 1H), 4.71 (d, 1H), 6.78 (d, 1H), 6.82 (s, 1H), 6.83 (d, 1H), 7.21 (t, 1H). LRMS: m/z 222 (M-H⁺). Analysis found C, 59.74; H, 7.98; N, 5.25%. C₁₃H₁₉NO₂.0.18H2O requires C, 59.82; H, 7.86; N, 5.37%. α_{D}=−5.66° (Methanol 10.6 mg/10 ml).

A sample of the title compound was re crystallised by vapour diffusion using a methanol: diethyl ether mix and an X-ray crystal structure obtained. The absolute stereochemistry of the title compound was determined from the diffraction data by the method of Flack (Acta Cryst. 1983, 439, 876-881) and was shown to have an (R)-configuration.

### Example 9

### 2-Amino-1-(3,5-dimethoxyphenyl)ethanol

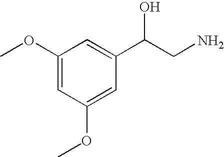

Prepared following the same method as for example 1 starting from 3,5-dimethoxybenzaldehyde (5.00 g, 0.03 mol). After refluxing in 6M HCl (aq) the reaction mixture was cooled and extracted with diethyl ether (2×80 ml). The organic layers were discarded and the aqueous layer basified by the addition of potassium carbonate. The aqueous residue was then extracted with ethyl acetate (3×70 ml). The organic extracts were combined and dried over anhydrous magnesium sulphate, filtered and concentrated in vacuo to give the title compound as a pale yellow oil (3.47 g, 0.018 mol, 59%). ¹H NMR (CD₃OD, 400 MHz) δ: 2.77-2.86 (m, 2H), 3.78 (s, 6H), 4.60 (m, 1H), 6.38 (s, 1H), 6.52 (s, 2H). LRMS: m/z 198 (M-H⁺).

### Example 10

### N-[2-(3,5-dimethoxyphenyl)-2-hydroxyethyl]propionamide

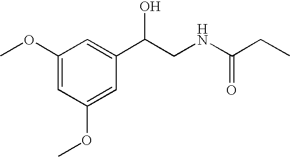

Prepared following the same method as for example 2 starting from the amine in example 9 (3.41 g, 0.017 mol). The crude reaction mixture was purified by column chromatography on silica eluting with dichloromethane:methanol (95:5) to give the title compound as a bright yellow oil (3.08 g, 0.012 mol, 70%). ¹H NMR (CDCl₃, 400 MHz) δ: 1.18 (m, 3H), 2.24 (m, 2H), 3.34 (m, 1H), 3.68 (m, 1H), 3.81 (s, 6H), 4.80 (dd, 1H), 5.95 (bs, 1H), 6.39 (s, 1H), 6.51 (s, 2H). LRMS: m/z 252 (M-H⁻).

### Example 11

### 1-(3,5-dimethoxyphenyl)-2-propylaminoethanol

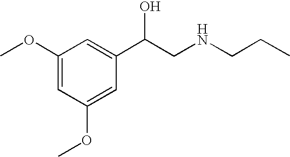

Prepared following the method as for example 3 starting from the amide in example 10 (3.06 g, 0.012 mol) to give the title compound as an orange oil (2.72 g, 0.01 μmol, 94%). ¹H NMR (CD₃OD, 400 MHz) δ: 0.95 (t, 3H), 1.56 (m, 2H), 2.61 (m, 2H), 2.77 (d, 2H), 3.78 (s, 6H), 4.70 (t, 1H), 6.38 (s, 1H), 6.51 (s, 2H). LRMS: m/z 240 (M-H⁺).

### Example 12

### 2-Chloro-N-[2-(3,5-dimethoxyphenyl)-2-hydroxyethyl]-N-propylacetamide

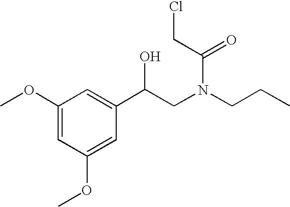

Prepared following the same method as for example 4 starting from the amine in example 11 (2.70 g, 0.01 μmol) to give the title compound as a yellow oil (3.56 g, 0.01 μmol. 100%). ¹H NMR (CDCl₃, 400 MHz) δ: 0.92 (t, 3H), 1.61 (m, 2H), 3.20 (m, 2H), 3.51-3.64 (m, 2H), 3.80 (d, 6H), 4.13 (s, 2H), 4.95 (m, 1H), 6.40 (m, 1H), 6.55 (s, 2H). LRMS: m/z 316 (M-H⁺).

### Example 13

### 6-(3,5-Dimethoxyphenyl)-4-propylmorpholin-3-one

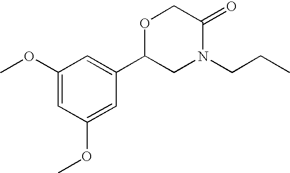

Prepared following the same method as for example 5 starting from the amide in example 12 (3.54 g, 0.011 mol) to give the title compound as a yellow oil (2.44 g, 0.09 mol, 78%). ¹H NMR (CDCl₃, 400 MHz) δ: 0.94 (t, 3H), 1.61 (m, 2H), 3.30 (m, 2H), 3.49 (m, 2H), 3.80 (s, 6H), 4.30 (d, 1H), 4.42 (d, 1H), 4.73 (dd, 1H), 6.42 (s, 1H), 6.53 (s, 2H). LRMS: m/z 280 (M-H⁺).

### Example 14

### 2-(3,5-Dimethoxyphenyl)-4-propylmorpholine

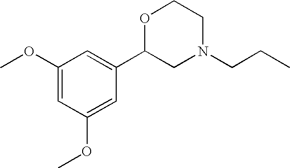

Prepared following the method as for example 6 starting from the amide in example 13 (2.42 g, 0.009 mol). After refluxing in 6M HCl (aq) the cooled reaction mixture was extracted with diethyl ether (2×80 ml). The organic layers were discarded and the aqueous basified by addition of potassium carbonate. The aqueous residue was then extracted with ethyl acetate (3×80 ml) and the organic extracts combined, dried over anhydrous magnesium sulphate, filtered then concentrated in vacuo to give the title compound as a pale orange oil (2.14 g, 0.008 mol, 93%). ¹H NMR (CD₃OD, 400 MHz) δ: 0.95 (t, 3H), 1.58 (m, 2H), 2.01 (m, 1H), 2.22 (dt, 1H), 2.38 (t, 2H), 2.83 (d, 1H), 2.93 (d, 1H), 3.78 (m, 7H), 4.01 (dd, 1H), 4.45 (dd, 1H), 6.39 (s, 1H), 6.49 (s, 2H). LRMS: m/z 266 (M-H⁺).

### Example 15A

### R-5-(4-Propylmorpholin-2-yl)benzene-1,3-diol

### Example 15B

### S-5-(4-Propylmorpholin-2-yl)benzene-1,3-diol

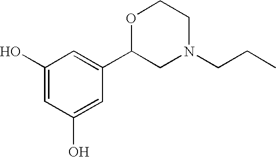

Prepared following the same route as for example 7 starting from the 3,5-dimethoxyphenyl compound in example 14 (1.00 g, 0.004 mol) giving the title racemic compound as a brown oil (145 mg, 0.61 mmol, 16%). The enantiomers were separated by chiral chromatography (Chiralpak AD 250*20 mm column) eluting with hexane: isopropyl alcohol: (80: 20) to give enantiomer 1 (15a) (5.2 mg) (ee>98.94%) and enantiomer 2 (15b) (5.1 mg) (ee>96.46%) as brown oils. Enantiomer 1 (15a): ¹H NMR (CD₃OD, 400 MHz) δ: 0.96 (t, 3H), 1.58 (m, 2H), 2.01 (t, 1H), 2.20 (dt, 1H), 2.37 (t, 2H), 2.81-2.92 (m, 2H), 3.89 (dt, 1H), 3.99 (dd, 1H), 4.38 (dd, 1H), 6.18 (t, 1H), 6.26 (s, 2H). LRMS: m/z 238 (M-H⁺). Enantiomer 2 (15b): ¹H NMR (CD₃OD, 400 MHz) δ: 0.95 (t, 3H), 1.58 (m, 2H), 2.01 (t, 1H), 2.20 (dt, 1H), 2.38 (t, 2H), 2.80-2.92 (q, 2H), 3.78 (dt, 1H), 3.98 (dd, 1H), 4.38 (dd, 1H), 6.18 (s, 1H), 6.25 (s, 2H). LRMS: m/z 238 (M-H⁺).

### Example 16

### 4-Fluoro-3-methoxybenzaldehyde

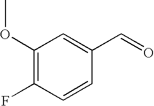

(4-Fluoro-3-methoxyphenyl)methanol (5.00 g, 0.03 mol) and manganese dioxide (33.4 g, 0.38 mol) were stirred in dichloromethane (100 ml) under an atmosphere of nitrogen, at gentle reflux for 16 hours. The cooled reaction mixture was then filtered through arbacel and concentrated in vacuo to give the title compound as a white solid (4.18 g, 0.027 mol, 85%). ¹H NMR (CDCl₃, 400 MHz) δ: 3.96 (s, 3H), 7.23 (d, 1H), 7.43 (m, 1H), 7.50 (d, 1H) 9.91 (s, 1H). Mpt: 61-63° C. Analysis found C, 62.18; H, 4.54%. C₈H₇FO₂ requires C, 62.34; H, 4.58%.

### Example 17

### 2-Amino-1-(4-fluoro-3-methoxyhenyl)ethanol

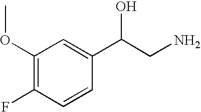

Prepared following the same method as for example 1 starting from 4-fluoro-3-methoxybenzaldehyde (4.17 g, 0.03 mol). After refluxing in 6M HCl (aq) the reaction mixture was cooled and extracted with diethyl ether (2×60 ml). The organic layers were discarded and the aqueous layer basified by the addition of potassium carbonate. The aqueous residue was then extracted with ethyl acetate (3×80 ml). The organic extracts were combined and dried over anhydrous magnesium sulphate, filtered and concentrated in vacuo to give the title compound as an orange oil (2.36 g, 0.013 mol, 47%). ¹H NMR (CD₃OD, 400 MHz) δ: 2.80-2.91 (m, 2H), 3.86 (s, 3H), 4.64 (m, 1H), 6.89 (m, 1H), 7.03 (t, 1H), 7.11 (dd, 1H). LRMS: m/z 186 (M-H⁺).

### Example 18

### N-[2-(4-Fluoro-3-methoxyphenyl)-2-hydroxyethyl]propionamide

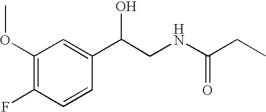

Prepared following the same method as for example 2 starting with the amine from example 17 (1.32 g, 0.007 mol). The crude reaction mixture was purified by column chromatography on silica eluting with ethyl acetate: pentane (2: 1) to give the title compound as a yellow oil that crystallised on standing (0.59 g, 0.002 mol, 35%). ¹H NMR (CDCl₃, 400 MHz) δ: 1.18 (t, 3H), 2.24 (q, 2H), 2.58 (bs, 1H), 3.34 (m, 1H), 3.63 (m, 1H), 3.88 (s, 3H), 4.82 (dd, 1H), 5.98 (bs, 1H), 6.82 (m, 1H), 7.01 (m, 2H). LRMS: m/z 242 (M-H⁺).

### Example 19

### 1-(4-Fluoro-3-methoxyphenyl)-2-propylaminoethanol

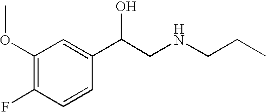

Prepared following the same method as for example 3 starting with the amide from example 18 (585 mg, 2.42 mmol). After refluxing in 6M HCl (aq) the reaction mixture was cooled and extracted with diethyl ether (2×50 ml). The organic layers were discarded and the aqueous layer basified by the addition of potassium carbonate. The aqueous residue was then extracted with ethyl acetate (3×50 ml). The organic extracts were combined and dried over anhydrous magnesium sulphate, filtered and concentrated in vacuo to give the title compound as a pale yellow oil (448 mg, 1.97 mmol, 81%). ¹H NMR (CD₃OD, 400 MHz) δ: 0.96 (t, 3H), 1.58 (m, 2H), 2.63 (m, 2H), 2.79 (d, 2H), 3.96 (s, 3H), 4.77 (t, 1H), 6.90 (m, 1H), 7.03 (t, 1H), 7.11 (d, 1H). LRMS: m/z 228 (M-H⁺).

### Example 20

### 2-Chloro-N-[2-(4-fluoro-3-methoxyphenyl)-2-hydroxyethyl]-N-propylacetamide

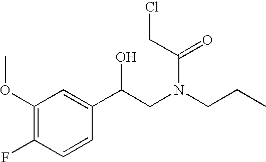

Prepared following the same method as for example 4 starting with the amine from example 19 (0.84 g, 4.00 mmol) to give the title compound as a yellow oil (0.97 g, 3.00 mmol, 87%). LRMS: m/z 304 (M-H⁺). This was taken on crude.

### Example 21

### 6-(4-Fluoro-3-methoxyphenyl)-4-propylmorpholin-3-one

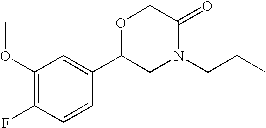

Prepared following the same method as for example 5 starting with the amide from example 20 (0.96 g, 3.00 mmol) to give the title compound as a yellow oil (0.64 g, 2.40 mmol, 75%). ¹H NMR (CDCl₃, 400 MHz) δ: 0.94 (t, 3H), 1.62 (m, 2H), 3.33 (m, 2H), 3.48 (m, 2H), 3.91 (s, 3H), 4.34 (d, 1H), 4.43 (d, 1H), 4.76 (dd, 1H), 6.85 (m, 1H), 7.01-7.08 (m, 2H). LRMS: m/z 268 (M-H⁺).

### Example 22

### 2-(4-Fluoro-3-methoxyphenyl)-4-propylmorpholine

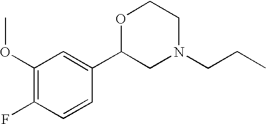

Prepared following the same method as for example 6 starting with the morpholin-3-one from example 21 (633 mg, 2.37 mmol). After refluxing in 6M HCl (aq) the reaction mixture was cooled and extracted with diethyl ether (2×20 ml). The organic layers were discarded and the aqueous layer basified by the addition of potassium carbonate. The aqueous residue was then extracted with ethyl acetate (3×20 ml). The organic extracts were combined and dried over anhydrous magnesium sulphate, filtered and concentrated in vacuo to give the title compound as a yellow oil (552 mg, 2.18 mmol, 92%). ¹H NMR (CD₃OD, 400 MHz) δ: 0.95 (t, 3H), 1.58 (m, 2H), 2.02 (t, 1H), 2.22 (dt, 1H), 2.38 (t, 2H), 2.85 (d, 1H), 2.93 (d, 1H), 3.80 (m, 1H), 3.84 (s, 3H), 4.01 (dd, 1H), 4.50 (dd, 1H), 6.88 (m, 1H), 7.02 (t, 1H), 7.09 (d, 1H). LRMS: m/z 254 (M-H⁺).

### Example 23A

### R-(+)-2-Fluoro-5-(4-propylmorpholin-2-yl)phenol

### Example 23B

### S—(−)-2-Fluoro-5-(4-propylmorpholin-2-yl)phenol

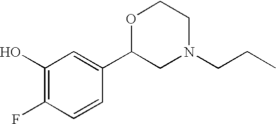

Prepared following the same method as for example 7 starting with the anisole from example 22 (200 mg, 0.789 mmol). The crude reaction mixture was purified by column chromatography on silica eluting with dichloromethane:methanol (90:10) to give the title racemic compound as a dark yellow viscous oil (149 mg, 0.62 mmol, 79%). The enantiomers were separated by chiral chromatography (Chiralpak AD 250*20 mm column) eluting with hexane: isopropyl alcohol: (90: 10) to give enantiomer 1 (23a) as an opaque oil (15 mg) (ee>99.5%) and enantiomer 2 (23b) as a crystalline solid (16 mg) (ee>99%). Enantiomer 1 (23a): ¹H NMR (CD₃OD, 400 MHz) δ: 0.95 (t, 3H), 1.58 (m, 2H), 2.01 (t, 1H), 2.21 (dt, 1H), 2.37 (t, 2H), 2.82-2.97 (bq, 2H), 3.78 (dt, 1H), 3.99 (dd, 1H), 4.43 (d, 1H), 6.78 (m, 1H), 6.89-7.01 (m, 2H). LRMS: m/z 240 (M-H⁺). α_{D}=+0.91 (Ethanol 1.10 mg/ml). Enantiomer 2 (23b): ¹H NMR (CD₃OD, 400 MHz) δ: 0.96 (t, 3H), 1.58 (m, 2H), 2.01 (t, 1H), 2.22 (dt, 1H), 2.38 (t, 2H), 2.78 (dd, 2H), 3.78 (dt, 1H), 4.00 (dd, 1H), 4.43 (dd, 1H), 6.78 (m, 1H), 6.91 (d, 1H), 6.98 (t, 1H). LRMS: m/z 240 (M-H⁺). α_{D}=−0.40 (Ethanol 1.00 mg/ml).

### Example 24

### 2-Amino-1-(4-benzyloxyphenyl)ethanol

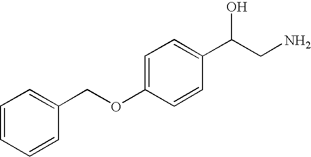

Potassium cyanide (20.15 g, 0.31 mol) and ammonium chloride (16.4 g, 0.31 mol) were dissolved in water (60 ml) to which was added 4-benzyloxybenzaldehyde (32.9 g, 0.155 mol) followed by diethyl ether (100 ml). The reaction mixture was stirred vigorously for 48 hours at room temperature before extracting with ethyl acetate (2×200 ml). The combined organic layers were dried over anhydrous magnesium sulphate, filtered and concentrated in vacuo to give the cyanohydrin intermediate as a yellow solid (34.2 g, 0.14 mol, 90%). The cyanohydrin was then dissolved in dry THF (300 ml) and borane-methyl sulphide complex (26.6 ml, 0.28 mol) was added. The reaction mixture was refluxed for 2 hours before being quenched with methanol (50 ml). Water (50 ml) was added followed by c.HCl (40 ml) and the reaction mixture was stirred for 2 hours until the exotherm subsided. The reaction mixture was then concentrated in vacuo and the residue diluted with water (100 ml). The aqueous solution was then basified by addition of NH₄OH (30 ml), and extracted with ethyl acetate (3×150 ml). The organic extracts were dried over anhydrous magnesium sulphate, filtered and concentrated in vacuo to give the title compound as a white solid (24.8 g, 0.10 mol, 73%). ¹H NMR (CDCl₃, 400 MHz) δ: 1.62 (bs, 3H), 2.81 (dd, 1H), 2.99 (d, 1H), 4.61 (q, 1H), 5.07 (s, 2H), 6.95 (d, 2H), 7.22-7.45 (m, 7H). LRMS: m/z 244 (M-H⁺).

### Example 25

### N-[2-(4-benzyloxylphenyl)-2-hydroxyethyl)propionamide

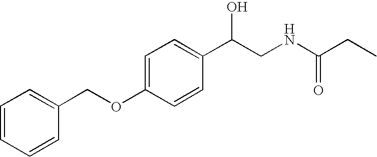

The amine from example 24 (24.8 g, 0.10 mol) was dissolved in dichloromethane (700 ml) and to this was added triethylamine (20.86 ml, 0.15 mol). The reaction mixture was stirred and cooled to 0° C., before propionyl chloride (7.12 ml, 0.082 mol) was added dropwise. The reaction mixture was then allowed to warm to room temperature over 16 hours before quenching with 3M HCl (aq) (20 ml) and water (100 ml). The reaction mixture was then extracted with dichloromethane (3×200 ml) and the combined organic layers dried over anhydrous magnesium sulphate, filtered and concentrated in vacuo to give the title compound as a clear viscous gum (27.5 g, 0.092 mol, 90%). ¹H NMR (CDCl₃, 400 MHz) δ: 1.10 (t, 3H), 2.19 (q, 2H), 3.32-3.43 (m, 4H), 4.81 (s, 2H), 5.11 (m, 1H), 6.99 (d, 2H), 7.25-7.42 (m, 7H). LRMS: m/z 298 (M-H⁻).

### Example 26

### 1-(4-benzyloxyphenyl)-2-propylaminoethanol

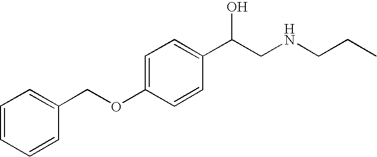

To the amide from example 25 (27.5 g, 0.092 mol) in dry THF (100 ml) was added borane-methyl sulphide complex (17.5 ml, 0.18 mol) and the reaction mixture was stirred at reflux for 2 hours. The reaction mixture was cooled then quenched with methanol (30 ml). Water (50 ml) and c.HCl (35 ml) were added and the reaction mixture stirred until all bubbling ceased before concentrating in vacuo. To the residue water (250 ml) was added, before basifying by addition of NH₄OH (30 ml). The aqueous layer was extracted with ethyl acetate (3×200 ml) and the combined organic extracts dried over anhydrous magnesium sulphate, filtered and concentrated in vacuo to give the title compound as a white solid (26.1 g, 00.9 mol, 99%). ¹H NMR (CD₃OD, 400 MHz) δ: 0.95 (t, 3H), 1.58 (q, 2H), 2.62 (m, 2H), 2.81 (m, 2H), 4.72 (dd, 1H), 5.05 (s, 2H), 6.95 (d, 2H), 7.24 (m, 3H), 7.35 (t, 2H), 7.41 (d, 2H). LRMS: m/z 286 (M-H⁺).

### Example 27

### 6-(4-benzyloxyphenyl)-4-propylmorpholin-3-one

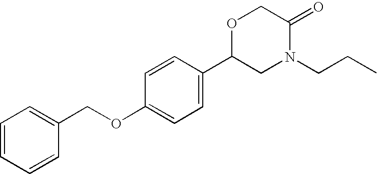

Sodium hydroxide (22.5 g, 0.56 mol) in water (100 ml) was added to the amine from example 26 (26.0 g, 0.09 mol) in dichloromethane (400 ml) and the solution vigorously stirred at room temperature. Chloroacetylchloride (8.6 ml, 0.11 mol) was then added and the reaction mixture stirred for a further 60 minutes. The layers were separated and the aqueous layer re-extracted with dichloromethane (200 ml). The organic extracts were combined, dried over anhydrous magnesium sulphate, filtered and concentrated in vacuo to give a colourless oil. Potassium hydroxide (15.0 g, 0.27 mol), isopropyl alcohol (400 ml) and the colourless oil residue were stirred together as an opaque solution with water (30 ml) for 2 hours. The reaction mixture was concentrated in vacuo and the yellow residue dissolved in ethyl acetate (200 ml). This was partitioned with water (200 ml) then brine (200 ml). The organic fraction was dried over anhydrous magnesium sulphate, filtered and concentrated in vacuo to give the title compound as a white solid (19.9 g, 0.06 mol, 67%). ¹H NMR (CDCl₃, 400 MHz) δ: 0.95 (t, 3H), 1.62 (m, 2H), 3.34 (m, 2H), 3.51 (m, 2H), 4.32 (d, 1H), 4.41 (d, 1H), 4.72 (dd, 1H), 5.04 (s, 2H), 6.98 (d, 2H), 7.31-7.43 (m, 7H). LRMS: m/z 326 (M-H⁺).

### Example 28

### 2-(4-benzyloxyphenyl)-4-propylmorpholine

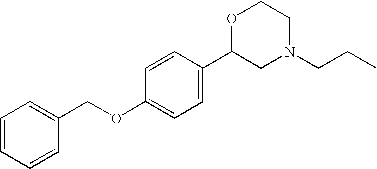

Prepared following the same method as for example 26 with the morpholin-3-one from example 27 (19.9 g, 0.061 mol) to give the title compound as a colourless oil (17 g, 0.055 mol, 90%). ¹H NMR (CDCl₃, 400 MHz) δ: 0.95 (t, 3H), 1.55 (q, 2H), 2.06 (t, 1H), 2.21 (dt, 1H), 2.35 (dd, 2H), 2.80 (d, 1H), 2.91 (d, 1H), 3.82 (dt, 1H), 4.02 (dd, 1H), 4.52 (dd, 1H), 5.05 (s, 2H), 6.98 (t, 2H), 7.24-7.42 (m, 7H). LRMS: m/z 312 (M-H⁺).

### Example 29

### 4-(4-Propylmorpholin-2-yl)phenol

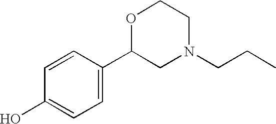

Benzyl ether from example 28 (3.0 g, 9.64 mmol) was dissolved in methanol (150 ml) and 10% palladium on charcoal (800 mg) was added. The reaction mixture was stirred for a few minutes before ammonium formate (6.17 g, 96.4 mmol) was added portionwise. The reaction mixture was carefully heated to 80° C. until gas evolution had ceased. After cooling, the reaction mixture was filtered through arbacel, washed with methanol (50 ml) and concentrated in vacuo to give the title compound as a white crystalline solid (1.51 g, 6.83 mmol, 71%). ¹H NMR (CDCl₃, 400 MHz) δ: 0.91 (t, 3H), 1.58 (q, 2H), 2.10 (t, 1H), 2.22 (t, 1H), 2.40 (dd, 2H), 2.81 (d, 1H), 2.93 (d, 1H), 3.85 (t, 1H), 4.02 (dd, 1H), 4.57 (d, 1H), 6.79 (d, 2H), 7.21 (d, 2H). LRMS: m/z 222 (M-H⁺).

### Example 30

### 2-Bromo-4-(4-Propylmorpholin-2-yl)phenol

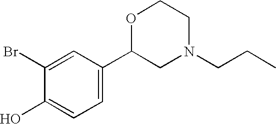

To the phenol from example 29 (200 mg, 0.9 mmol) in dichloromethane (5 ml) was added N-bromosuccinimide (161 mg, 0.9 mmol). The reaction mixture was stirred at room temperature for 55 hours, before concentrating in vacuo. The crude product was purified by column chromatography on silica eluting with dichloromethane:methanol (95:5) to give the title compound as a white foam (117.5 mg, 0.39 mmol, 44%). ¹H NMR (CDCl₃, 400 MHz) δ: 0.96 (t, 3H), 1.59 (q, 2H), 2.03 (t, 1H), 2.23 (t, 1H), 2.40 (t, 2H), 2.81 (d, 1H), 2.98 (d, 1H), 3.82 (t, 1H), 4.01 (d, 1H), 4.56 (d, 1H), 6.96 (d, 1H), 7.20 (d, 1H), 7.49 (s, 1H). LRMS: m/z 302 (M-H⁺, Br isotope).

### Example 31

### 2-(4-benzyloxy-3-bromophenyl)-4-propylmorpholine

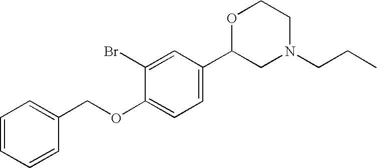

To the phenol from example 30 (117.5 mg, 0.39 mmol) in dry DMF (10 ml), under an atmosphere of nitrogen, was added potassium carbonate (75 mg, 0.54 mmol) and benzyl bromide (0.07 ml, 0.54 mmol). The reaction mixture was heated to 150° C. for 48 hours. After cooling, the reaction mixture was concentrated in vacuo and the residue partitioned between ethyl acetate (50 ml) and water (50 ml). The aqueous layer was then re-extracted with ethyl acetate (2×20 ml). The combined organic extracts were then dried over anhydrous magnesium sulphate, filtered and concentrated in vacuo to give the crude product as a brown oil. This was purified by column chromatography on silica eluting with dichloromethane:methanol (98:2) to give the title compound as a colourless oil (153 mg, 0.39 mmol, 100%). ¹H NMR (CDCl₃, 400 MHz) δ: 0.93 (t, 3H), 1.56 (q, 2H), 2.05 (t, 1H), 2.25 (t, 1H), 2.37 (t, 2H), 2.82 (d, 1H), 2.92 (d, 1H), 3.85 (t, 1H), 4.02 (d, 1H), 4.52 (d, 1H), 5.15 (s, 2H), 6.87 (d, 1H), 7.20 (d, 1H), 7.30 (d, 1H), 7.37 (t, 2H), 7.45 (d, 2H), 7.58 (s, 1H). LRMS: m/z 392 (M-H⁺).

### Example 32

### 2-Benzyloxy-5-(4-propylmorpholin-2-yl)benzoic acid methyl ester

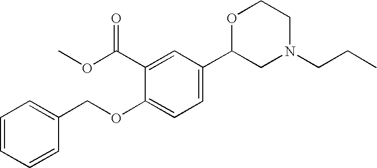

To the bromide from example 31 (153 mg, 0.39 mmol) in dry DMF (4 ml) was added triethylamine (2.1 ml, 0.78 mmol) and methanol (2 ml) and the reaction mixture stirred for 5 minutes. [1,1′-Bis(diphenylphosphino)ferrocene]dichloropalladium (II), complex with dichloromethane (1:1) (16 mg, 0.02 mmol) was added before carbon monoxide (9) (3 inflated balloons) was bubbled through the reaction mixture. The reaction mixture was then heated to 100° C. for 16 hours under an atmosphere of carbon monoxide. After cooling, the reaction mixture was concentrated in vacuo and the residue partitioned between ethyl acetate (25 ml) and water (20 ml). The organic layer was separated, washed with brine (20 ml) and dried over anhydrous magnesium sulphate, filtered and concentrated in vacuo to give a black solid. Purification by column chromatography on silica eluting with dichloromethane: methanol: ammonia (90:10:1) gave the title compound as a colourless oil (105 mg, 0.28 mmol, 73%). ¹H NMR (CDCl₃, 400 MHz) δ: 0.94 (t, 3H), 1.60 (m, 2H), 2.18 (s, 4H), 2.43 (m, 2H), 3.00 (m, 2H), 3.90 (s, 3H), 4.04 d, 1H), 5.18 (s, 2H), 5.97 (d, 1H), 7.26-7.47 (m, 6H), 7.82 (s, 1H). LRMS: m/z 370 (M-H⁺).

### Example 33

### 2-Benzyloxy-5-(4-propylmorpholin-2-yl)benzoic acid

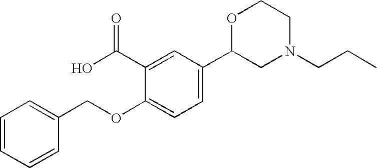

To the methyl ester from example 32 (105 mg, 0.28 mmol) in methanol (5 ml) was added 10% sodium hydroxide (aq) (15 ml) and the milky white suspension was refluxed for 2 hours. The now colourless reaction mixture was cooled then neutralised by addition of 2M HCl (aq) (few drops). The reaction mixture was then concentrated in vacuo to give the title compound as an off-white solid (99 mg, 0.28 mmol, 100%). LRMS: m/z 355 (M-H⁺). This material was taken on crude to example 34.

### Example 34

### 2-Benzyloxy-5-(4-propylmorpholin-2-yl)benzamide

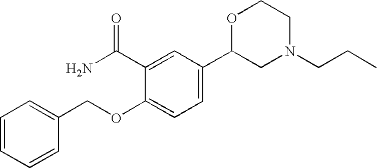

To the crude benzoic acid from example 33 (99 mg, 0.28 mmol) was added thionyl chloride (5 ml) and the reaction mixture heated to 50° C. for 2 hours. The reaction mixture was cooled and the excess thionyl chloride was removed in vacuo. The residue was then dissolved in dichloromethane (10 ml) and ammonia (g) was bubbled through the reaction mixture for 10 minutes. The resulting suspension was stirred at room temperature for 1 hour before concentrating in vacuo. The crude material was purified by column chromatography on silica eluting with dichloromethane: methanol: ammonia (95:5:0.5) to give the title compound as an off-white solid (88 mg, 0.25 mmol, 90%). ¹H NMR (CDCl₃, 400 MHz) δ: 0.94 (t, 3H), 1.59 (m, 2H), 2.15-2.42 (m, 4H), 2.87 (m, 1H), 3.03 (m, 1H), 3.96 (m, 1H), 4.02 (d, 1H), 4.67 (m, 1H), 5.19 (s, 2H), 5.72 (m, 1H), 7.04 (d, 1H), 7.41 (m, 5H), 7.50 (d, 1H), 7.70 (m, 1H), 8.21 (s, 1H). LRMS: m/z 355 (M-H⁺).

### Example 35

### 2-Hydroxy-5-(4-propylmorpholin-2-yl)benzamide

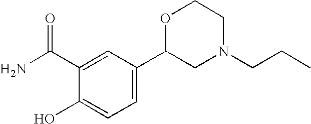

Prepared using the same method as for example 29 with the benzyl ester from example 34 (80 mg, 0.22 mmol) to give the title compound as an off-white solid (56 mg, 0.21 mmol, 96%). ¹H NMR (CD₃OD, 400 MHz) δ: 0.95 (t, 3H), 1.55 (m, 2H), 2.13 (t, 1H), 2.29 (t, 1H), 2.42 (m, 2H), 2.88 (d, 1H), 2.97 (d, 1H), 3.81 (t, 1H), 4.00 (d, 1H), 4.49 (d, 1H), 6.87 (d, 1H), 7.42 (d, 1H), 7.78 (s, 1H). LRMS: m/z 265 (M-H⁺).

### Example 36

### 2-Nitro-4-(4-propylmorpholin-2-yl)phenol

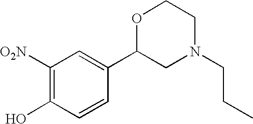

The phenol from example 29 (100 mg, 0.45 mmol) was dissolved in nitric acid:water (1:3) (2 ml) and stirred at room temperature for 10 minutes. The reaction mixture was then diluted with water (5 ml) and basified with NH₄OH (1 ml), before extracting into ethyl acetate (3×10 ml). The organic extracts were combined and dried over anhydrous magnesium sulphate, filtered and concentrated in vacuo to give the title compound as a yellow solid (95 mg, 0.35 mmol, 79%). ¹H NMR (CDCl₃, 400 MHz) δ: 0.97 (t, 3H), 1.33 (t, 2H), 1.43-1.79 (bm, 4H), 2.02 (d, 3H), 4.06 (m, 2H), 7.17 (d, 1H), 7.60 (d, 1H), 8.16 (s, 1H), 10.55 (bs, 1H). LRMS: m/z 267 (M-H⁺).

### Example 37

### 2-Amino-4-(4-propylmorpholin-2-yl)phenol

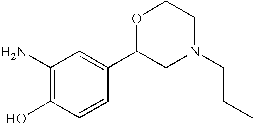

To the nitro from example 36 (95 mg, 0.35 mmol) in ethanol (10 ml) was added 10% palladium on charcoal (50 mg) and ammonium formate (100 mg, XS). The reaction mixture was gently heated to 70° C. and held at this temperature for 1 hour before it was allowed to cool to room temperature. The reaction mixture was then filtered through arbacel and washed with ethanol (20 ml) then dichloromethane (20 ml). The organic washes were combined and concentrated in vacuo to give the title compound as a yellow solid (65 mg, 0.28 mmol, 78%). ¹H NMR (CDCl₃, 400 MHz) δ: 0.91 (t, 3H), 1.55 (m, 2H), 2.12 (t, 1H), 2.25 (dt, 1H), 2.40 (t, 2H), 2.81-2.92 (dd, 2H), 3.82 (t, 1H), 4.00 (d, 1H), 4.42 (d, 1H), 6.60 (m, 2H), 6.71 (s, 1H). LRMS: m/z 237 (M-H⁺).

### Example 38

### 5-Bromo-2-(2,5-dimethylpyrrol-1-yl pyridine

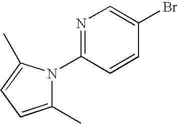

5-Bromopyridin-2-yl-amine (13.8 g, 0.08 mol), acetonylacetone (14.1 ml, 0.12 mol) and p-toluenesulphonic acid (100 mg) were dissolved in toluene (180 ml) and refluxed under Dean Stark conditions for 14 hours. After cooling, the brown solution was poured into water (200 ml) and extracted with toluene (2×200 ml). The organic extracts were combined and washed with brine (50 ml) then dried over anhydrous magnesium sulphate, filtered and concentrated in vacuo to give crude product. This was purified by column chromatography on silica eluting with ethyl acetate: pentane (1:3) to give the title compound as a brown oil (18.4 g, 0.073 mol, 92%). ¹H NMR (CDCl₃, 400 MHz) δ: 2.18 (s, 6H), 5.90 (s, 2H), 7.11 (d, 1H), 7.92 (d, 1H), 8.62 (s, 1H). LRMS: m/z 253 (M-H′, Br isotope).

### Example 39

### 2-Chloro 1-[6-(2,5-dimethylpyrrol-1-yl)pyridin-3-yl]ethanone

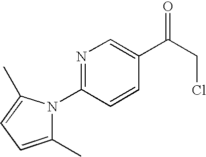

To a solution of bromo pyridine from example 38 (2 g, 8.0 mmol) at −78° C., in dry THF (30 ml), was added butyllithium (2.5M in hexanes) (3.5 ml 8.8 mmol), dropwise over 20 minutes. The reaction mixture was stirred for 30 minutes then 2-chloro-N-methoxy-N-methylacetamide (1.2 g, 8.8 mmol) in dry THF (20 ml) was added dropwise keeping the temperature at −78° C. Stirring was continued for 30 minutes at this temperature before 1M HCl (aq) (50 ml) was added and the reaction mixture warmed to room temperature. The organic layer was separated and the aqueous layer washed with ethyl acetate (50 ml). The organic layers were combined then washed with 3M NaOH (aq) (10 ml) and brine (10 ml) before being dried over anhydrous magnesium sulphate, filtered and concentrated in vacuo to give crude title compound as a brown oil (1.34 g, 5.4 mmol, 67%). ¹H NMR (CDCl₃, 400 MHz) δ: 2.20 (s, 6H), 4.68 (s, 2H), 5.92 (s, 2H), 7.32 (d, 1H), 8.38 (d, 1H), 9.16 (s, 1H). LRMS: m/z 249 (M-H—).

### Example 40

### 2-(2,5-dimethylpyrrol-1-yl)-5-oxiranylpyridine

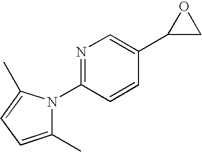

To the ketone from example 39 (1.34 g, 5.4 mmol) dissolved in dry THF (20 ml), cooled to 0° C., was added sodium borohydride (308 mg, 8.1 mmol) portionwise. The reaction mixture was stirred for 2 hours then 3M NaOH (aq) (10 ml) was added and stirring continued for a further 16 hours. The reaction mixture was extracted with ethyl acetate (2×20 ml) and the combined organic extracts washed with brine (5 ml), dried over anhydrous magnesium sulphate, filtered and concentrated it) vacuo. The residue was purified by column chromatography on silica eluting with ethyl acetate: pentane (1:5) to give the title compound as a colourless oil (900 mg, 4.2 mmol, 78%). ¹H NMR (CDCl₃, 400 MHz) δ: 2.13 (s, 6H), 2.91 (dd, 1H), 3.25 (t, 1H), 3.98 (t, 1H), 5.90 (s, 2H), 7.20 (d, 1H), 7.62 (dd, 1H), 8.58 (s, 1H). LRMS: m/z 215 (M-H⁺).

### Example 41

### 1-[6-(2,5-dimethylpyrrol-1-yl)pyridin-3-yl]-2-propylaminoethanol

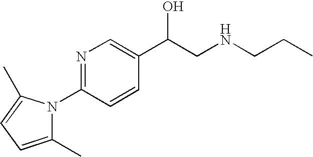

To the epoxide from example 40 (900 mg, 4.2 mmol) in DMSO (5 ml) was added propylamine (4 ml, 4.8 mmol) and the reaction mixture was heated to 40° C. for 4 days. The reaction mixture was then cooled and 3M HCl (aq) (10 ml) and water (10 ml) were added before washing with diethyl ether (2×10 ml). This organic layer was discarded. The aqueous layer was basified with NH₄OH (5 ml) and extracted with ethyl acetate (3×10 ml). The organic extracts were combined and dried over anhydrous magnesium sulphate, filtered and concentrated in vacuo to give the title compound as an oil (1.15 g, 4.2 mmol, 100%). ¹H NMR (CDCl₃, 400 MHz) δ: 0.93 (t, 3H), 1.62 (m, 2H), 2.11 (s, 6H), 2.69-2.82 (m, 3H), 3.06 (dd, 1H), 3.60 (bs, 2H), 4.92 (dd, 1H), 5.84 (s, 2H), 7.20 (d, 1H), 7.88 (d, 1H), 8.61 (s, 1H). LRMS: m/z 274 (M-H⁺).

### Example 42

### 6-[6-(2,5-dimethylpyrrol-1-yl)pyridin-3-yl]-4-propylmorpholi-3-one

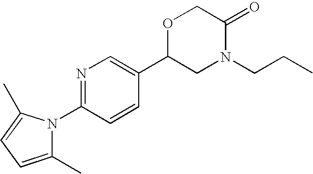

Prepared following the same method as for example 27 with the amine from example 41 (1.15 g, 4.2 mmol). Purification by column chromatography on silica eluting with dichloromethane:methanol (98:2) gave the title compound as a brown film (191 mg, 0.61 mmol, 14%). ¹H NMR (CDCl₃, 400 MHz) δ: 0.97 (t, 3H), 1.65 (m, 2H), 2.13 (s, 6H), 3.38 (m, 1H), 3.42-3.56 (m, 2H), 6.61 (t, 1H), 4.35 (d, 1H), 4.45 (d, 1H), 4.91 (dd, 1H), 6.91 (s, 2H), 7.22 (d, 1H), 7.89 (d, 1H), 8.61 (s, 1H). LRMS: m/z 314 (M-H⁺).

### Example 43

### 6-[6-(2,5-dimethylpyrrol-1-yl)pyridin-3-yl]-4-propylmorpholine

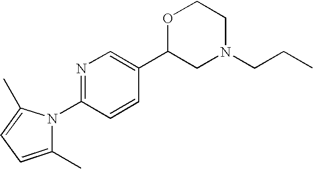

To a solution of the morpholin-3-one from example 42 (191 mg, 0.61 mmol) in dry THF (5 ml) was added lithium aluminum hydride (1M solution in diethyl ether) (1.25 ml, 0.61 mmol) and the reaction mixture was warmed to reflux for 2.5 hours. The reaction mixture was cooled to room temperature then 1M NaOH (1.25 ml) was added to give a white precipitate. The reaction mixture was filtered and concentrated in vacuo. The white solid was discarded. The concenrated filtrate was purified by column chromatography on silica eluting with dichloromethane:methanol (95:5) to give the title compound as a white film (108 mg, 0.36 mmol, 59%). ¹H NMR (CDCl₃, 400 MHz) δ: 0.92 (t, 3H), 1.61 (q, 2H), 2.10 (s, 6H), 2.15 (m, 1H), 2.29 (dt, 1H), 2.40 (t, 2H), 2.82 (d, 1H), 3.02 (d, 1H), 3.90 (t, 1H), 4.08 (d, 1H), 4.71 (d, 1H), 5.89 (s, 2H), 7.20 (d, 1H), 7.81 (d, 1H), 8.60 (s, 1H). LRMS: m/z 300 (M-H⁺).

### Examples 44A and 44B

### 5-(4-propylmorpholin-2-yl)pyridin-2-ylamine

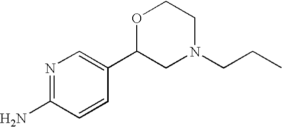

To the 2,5-dimethylpyrrole from example 43 (45 mg, 0.15 mmol) in ethanol (3 ml) was added hydroxylamine hydrochloride (52 mg, 0.75 mmol) and the reaction mixture heated to 80° C. for 20 hours. The reaction mixture was cooled to room temperature and concentrated in vacuo. The residue was purified by column chromatography on silica eluting with dichloromethane: methanol: ammonia (90:10:1) to give the racemic compound as a colourless film (31 mg, 0.14 mmol, 94%). ¹H NMR (CDCl₃, 400 MHz) δ: 0.92 (t, 3H), 1.60 (m, 2H), 2.11 (t, 1H), 2.25 (dt, 1H), 2.41 (t, 2H), 2.82-2.91 (dd, 2H), 3.89 (dt, 1H), 4.01 (dd, 1H), 4.57 (bd, 3H), 6.49 (d, 1H), 7.42 (d, 1H), 8.02 (s, 1H). LRMS: m/z 222 (M-H⁺).

A sample of this racemic product (580 mg) was separated into it's constituent enantiomers by chiral HPLC Conditions used: Chiralpak AD column (250×21.2 mm), Eluent methanol: ethanol (1:1), flow rate 15 mL/min.

The faster eluting enantiomer Example 44A (retention time 8.3 min) was obtained in >99% ee

¹H NMR (CDCl₃, 400 MHz) was identical to that of the racemate. LRMS: m/z 222. Analysis found C, 63.54; H, 8.60; N, 18.38%. C₁₂H₁₉N₃O.3H₂O requires C, 63.58; H, 8.71; N, 18.53%.

[α]₅₄₆²⁵−2.1 (c=0.12, MeOH); [α]₄₃₆²⁵−8.9 (c=0.12, MeOH)

The slower eluting enantiomer, Example 44B (retention time 9.4 min) was obtained in 98.9% e.e.

¹H NMR (CDCl₃, 400 MHz) was identical to that of the racemate. LRMS: m/Z 222. Analysis found C, 63.53; H, 8.57; N, 18.36%. C₁₂H₁₉N₃O.3H₂O requires C, 63.58; H, 8.71; N, 18.53%.

[α]₅₄₆²⁵+2.4 (c=0.12, MeOH); [α]₄₃₆²⁵+7.2 (c=0.12, MeOH)

### Example 45

### 2-Ethyl-6-(3-methoxy-phenyl)-4-propyl-morpholin-3-one

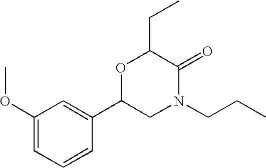

Sodium hydroxide (0.48 g, 12.0 mmol) in water (2 mL) was added to the product from example 3 (0.50 g, 2.4 mmol) in dichloromethane (5 mL) and the mixture stirred at room temperature. 2-Chlorobutyryl chloride (0.28 mL, 2.87 mmol) was then added dropwise and the reaction mixture stirred for 60 hours. The reaction mixture was diluted with dichloromethane (10 mL) and the aqueous layer was separated. The organic layer was dried over anhydrous magnesium sulphate, filtered and concentrated in vacuo to give the crude product as a clear oil (contained mixture of cyclised and uncyclised material) (0.57 g). LRMS: m/z 314 (M-H⁺ of uncyclised material), 296 (M-H⁺ less water), 278 (M-H⁺ of cyclised product). Potassium hydroxide (0.13 g, 2.20 mmol) was dissolved in water (1 mL) and added to a solution of the crude product (0.57 g, 1.83 mmol) in isopropyl alcohol (5 mL). The reaction mixture was stirred at room temperature overnight and the organic solvent then evaporated in vacuo. The residue was dissolved in ethyl acetate (10 mL) and the aqueous layer separated. The organic layer was dried over anhydrous magnesium sulphate, filtered and concentrated in vacuo to give the crude product as an oil. The residue was purified by column chromatography on silica eluting with ethyl acetate:pentane (1:5 to 1:1) to give the title compound as a clear oil (326 mg, 1.17 mmol, 49%) as a mixture of diastereomers. ¹H NMR (CDCl₃, 400 MHz) δ: 0.90 (t, 3H), 1.00 (t, 3H), 1.60 (m, 2H), 2.00 (bm, 2H), 3.10-3.60 (m, 4H), 3.80 (s, 3H), 4.20 (d, 0.5H), 4.25 (d, 0.5H), 4.75 (d, 0.5H), 4.90 (d, 0.5H), 6.80 (d, 1H), 6.90 (m, 2H), 7.25 (m, 1H). LRMS (APCI): m/z 278 (MH⁺), 276 (MH⁻).

### Examples 46A and 46B

### 2-Ethyl-6-(3-methoxy-phenyl)-4-propyl-morpholine

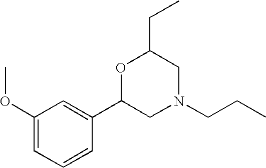

Borane-tetrahydrofuran complex (1M in THF) (3 mL, 3 mmol) was added dropwise to the product from example 45 (0.33 g, 1.18 mmol) in dry THF (4 mL) under an atmosphere of nitrogen. The reaction mixture was heated at 85° C. for 3 hours then cooled and quenched by the addition of methanol (1 mL). The reaction mixture was then concentrated in vacuo and the residue suspended in 6N HCl (aq) (10 mL) and heated to 60° C. for 1.5 hours. The reaction mixture was cooled and extracted with diethyl ether (2×10 mL). The aqueous layer was rendered basic (pH 9-10) by addition of solid potassium carbonate before re-extracting with dichloromethane (2×15 mL). The dichloromethane extracts were dried over anhydrous magnesium sulphate, filtered and concentrated in vacuo to give the crude products as a clear oil. Purification by column chromatography on silica eluting with ethyl acetate: pentane (1:10) yielded the two title compounds as single diastereomers.

Example 46A: clear oil (0.10 g, 0.38 mmol, 32%): ¹H NMR (CDCl₃, 400 MHz) δ: 1.00 (m 6H), 1.60 (bm, 3H), 1.85 (m, 1H), 2.25 (bt, 2H), 2.35 (s, 1H), 2.45 (m, 1H), 2.60 (m, 1H), 2.65 (m, 1H), 3.70 (s, 1H), 3.80 (s, 3H), 4.80 (s, 1H), 6.80 (d, 1H), 7.00 (m, 2H), 7.25 (m, 1H). LRMS (APCI): m/z 264 (M-H⁺).

Example 46B: clear oil (0.10 g, 0.38 mmol, 32%): ¹H NMR (CDCl₃, 400 MHz) δ: 0.90 (t, 3H), 1.00 (t, 3H), 1.60 (bm, 4H), 1.80 (bs, 1H), 2.00 (bs, 1H), 2.35 (bs, 2H), 2.85 (bd, 1H), 2.95 (bd, 1H), 3.60 (s, 1H), 3.80 (s, 3H), 4.60 (s, 1H), 6.80 (d, 1H), 6.95 (s, 2H), 7.25 (t, 1H). LRMS (APCI): m/z 264 (MH⁺).

### Example 47A

### 3-(6-Ethyl-4-propyl-morpholin-2-yl)-phenol

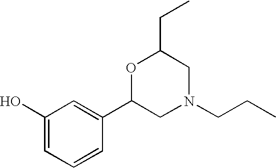

Hydrobromic acid (48% aq., 5 mL) and the product from example 46A (0.10 g, 0.38 mmol) were heated at 80° C. for 16 hours. After cooling the reaction mixture was concentrated in vacuo. The residue was partitioned between aqueous ammonia (0.880, 15 mL) and dichloromethane (15 mL), the layers were separated and the aqueous layer re-extracted with dichloromethane (2×15 mL). The organic extracts were combined, dried over anhydrous magnesium sulphate, filtered and concentrated in vacuo. The crude product was purified by column chromatography on silica eluting with dichloromethane, then dichloromethane:methanol (99:1 to 95:5) to yield the title compound as a clear oil (65 mg, 0.26 mmol, 69%) as the single diastereoisomer. ¹H NMR (CDCl₃, 400 MHz) δ: 0.95 (m 6H), 1.60 (m, 3H), 1.85 (m, 1H), 2.25 (m, 2H), 2.45 (m, 2H), 2.55 (q, 1H), 2.75 (d, 1H), 3.75 (s, 1H), 4.80 (m, 1H), 6.70 (d, 1H), 6.90 (s, 1H), 7.00 (1H, d), 7.25 (t, 1H). LRMS (APCI): m/z 250 (MH⁺). Analysis found C, 70.94%; H, 9.16%; N, 5.53%. C₁₅H₂₃NO₂.0.3H₂0 requires C, 70.72%; H, 9.34%; N, 5.50%.

### Example 47B

### 3-(6-Ethyl-4-propyl-morpholin-2-yl)-phenol

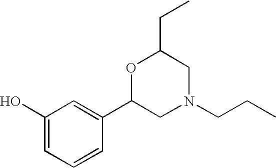

Prepared following the same method as for example 47A with the product from example 46B (0.10 g, 0.38 mmol). Purification by column chromatography on silica was not required. The title compound was obtained as a yellow oil (57 mg, 0.23 mmol, 60%) as the single diastereoisomer. ¹H NMR (CDCl₃, 400 MHz) δ: 0.90 (t, 3H), 1.00 (t, 3H), 1.60 (m, 4H), 1.85 (t, 1H), 2.00 (t, 1H), 2.35 (m, 2H), 2.90 (d, 1H), 3.00 (d, 1H), 3.65 (m, 1H), 4.60 (m, 1H), 6.75 (d, 1H), 6.80 (s, 1H), 6.90 (1H, d), 7.20 (t, 1H). LRMS (ESI): m/z 250 (MH⁺), 248 (M-H⁻). Analysis found C, 71.63%; H, 9.19%; N, 5.55%. C₁₅H₂₃NO₂.0.1H₂O requires C, 71.73%; H, 9.31%; N, 5.58%.

### Example 48

### 2-Methyl-6-(3-methoxy-phenyl)-4-propyl-morpholin-3-one

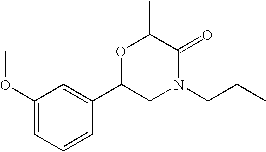

Prepared following the same method as for example 45 with the product from example 3 (0.44 g, 2.10 mmol) and 2-chloropropionyl chloride (0.25 mL. 2.50 mmol). Purification by column chromatography on silica of the title compound was not required. The title compound was obtained as a clear oil (0.42 g, 1.60 mmol, 76%) as a mixture of diastereomers. ¹H NMR (CDCl₃, 400 MHz) δ: 0.95 (t, 3H), 1.60 (m, 5H), 3.30 (bm, 2H), 3.50 (bm, 2H), 3.80 (s, 3H), 4.40 (q, 0.5H), 4.55 (q, 0.5H), 4.80 (dd, 0.5H), 4.95 (dd, 0.5H), 6.85 (d, 1H), 6.95 (s, 2H), 7.25 (m, 1H). LRMS (APCI): m/z 264 (MH⁺), 262 (MH⁻).

### Example 49A and 49B

### 2-Methyl-6-(3-methoxy-phenyl)-4-propyl-morpholine

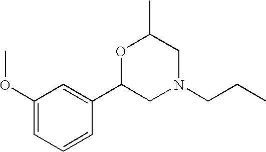

Prepared following the same method as for example 46 with the product from example 48 (0.42 g, 1.6 mmol). Purification by column chromatography on silica eluting with ethyl acetate: pentane (1:6) yielded the two title compounds as single diastereomers.

Example 49A: clear oil (0.10 g, 0.40 mmol, 25%): ¹H NMR (CDCl₃, 400 MHz) δ: 0.95 (t 3H), 1.30 (d, 3H), 1.60 (m, 2H), 2.20-2.35 (m, 3H), 2.50 (d, 1H), 2.60 (m, 1H), 2.65 (d, 1H), 3.80 (s, 3H), 4.00 (s, 1H), 4.85 (s, 1H), 6.80 (d, 1H), 7.05 (m, 2H), 7.25 (m, 1H). LRMS (APCI): m/z 250 (MH⁺).

Example 49B: clear oil (0.10 g, 0.40 mmol, 25%): ¹H NMR (CDCl₃, 400 MHz) δ: 0.90 (t 3H), 1.25 (m, 3H), 1.60 (m, 2H), 1.80 (m, 1H), 2.00 (bm, 1H), 2.35 (s, 2H), 2.80 (d, 1H), 2.90 (d, 1H), 3.80 (s, 3H), 3.85 (s, 1H), 4.60 (s, 1H), 6.80 (d, 1H), 7.00 (m, 2H), 7.25 (m, 1H). LRMS (APCI): m/z 250 (MH⁺).

### Example 50A

### 3-(6-Methyl-4-propyl-morpholin-2-yl)-phenol

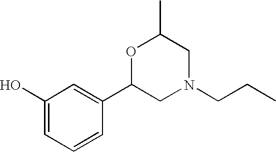

Prepared following the same method as for example 47A with the product from example 49A (0.10 g, 0.4 mmol). Purification by column chromatography on silica eluting with dichloromethane, then dichloromethane:methanol (99:1) yielded the title compound as a clear oil (70 mg, 0.30 mmol, 74%) as the single diastereoisomer. ¹H NMR (CDCl₃, 400 MHz) δ: 0.95 (t, 3H), 1.35 (d, 3H), 1.55 (m, 2H), 2.25 (m, 2H), 2.35 (m, 1H), 2.50 (m, 1H), 2.55 (m, 1H), 2.75 (d, 1H), 4.05 (s, 1H), 4.85 (m, 1H), 6.70 (d, 1H), 6.90 (s, 1H), 7.00 (1H, d), 7.20 (t, 1H). LRMS (APCI): m/z 236 (MH⁺). Analysis found C, 70.62%; H, 8.89%; N, 5.95%. C₁₄H₂₁NO₂.0.1H₂0 requires C, 70.91%; H, 9.01%; N, 5.91%.

### Example 50B

### 3-(6-Methyl-4-propyl-morpholin-2-yl)-phenol

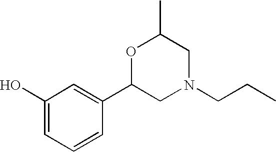

Prepared following the same method as for example 47A with the product from example 49B (0.10 g, 0.4 mmol). Purification by column chromatography on silica was not required. The title compound was obtained as a yellow oil (100 mg, 0.42 mmol, 103%—contained 3% starting material) as the single diastereomer. ¹H NMR (CDCl₃, 400 MHz) δ: 0.90 (t, 3H), 1.25 (d, 3H), 1.60 (m, 2H), 1.85 (m, 1H), 2.00 (m, 1H), 2.35 (m, 2H), 2.85 (d, 1H), 3.00 (d, 1H), 3.85 (s, 1H), 4.60 (d, 1H), 6.75 (d, 1H), 6.80 (s, 1H), 6.90 (1H, d), 7.20 (m, 1H). LRMS (APCI): m/z 236 (MH⁺). Analysis found C, 69.38%; H, 8.86%; N, 5.73%. C₁₄H₂₁NO₂.0.45H₂0 requires C, 69.33%; H, 9.06%; N, 5.78%.

### Example 51

### 1-(4-Chloro-3-methoxy-phenyl)-2-propylamino-ethanol

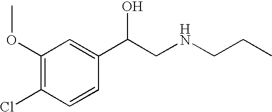

Sodium triacetoxyborohydride (1.25 g, 5.89 mmol) was added with care to a solution of 2-amino-1-(4-chloro-3-methoxy-phenyl)-ethanol (J. Med. Chem., 30(10), 1887, (1987)) (600 mg, 2.98 mmol) and propionaldehyde (0.22 mL, 2.96 mmol) in dichloromethane (10 mL), and the reaction mixture was stirred at room temperature for 1 hour. Sodium bicarbonate solution (sat. aq., 10 mL) was added dropwise and then the reaction mixture was diluted further with water (20 mL) and dichloromethane (20 mL). The aqueous layer was separated and re-extracted with dichloromethane (2×20 mL). The combined organic layers were dried over anhydrous magnesium sulphate, filtered and concentrated in vacuo. The crude product was purified by column chromatography on silica eluting with dichloromethane:methanol:0.880 ammonia (95:5:0.5 to 92:8:0.8) to yield the title compound as a solid (320 mg, 1.31 mmol, 44%). ¹H NMR (CDCl₃, 400 MHz) δ: 0.90 (t, 3H), 1.50 (q, 2H), 2.50-2.70 (m, 5H), 2.90 (dd, 1H), 3.80 (s, 3H), 4.65 (dd, 1H), 6.85 (d, 1H), 7.00 (1H, d), 7.30 (bd, 1H). LRMS (APCI): m/z 244 (MH⁺), 226 (MHz less H₂O).

### Example 52

### 6-(4-Chloro-3-methoxy-phenyl)-4-propyl-morpholin-3-one

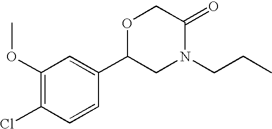

Chloroacetyl chloride (0.11 mL, 1.33 mmol) was added to a solution of the product from example 51 (0.31 g, 1.27 mmol) and triethylamine (0.19 mL, 1.36 mmol) in dichloromethane (10 mL) and stirred at room temperature for 60 hours. The reaction mixture was diluted with dichloromethane (20 mL) and washed with hydrochloric acid (aq. 1N, 10 mL), water (10 mL) and sodium bicarbonate solution (sat. aq., 10 mL). The organic layer was dried over anhydrous magnesium sulphate, filtered and concentrated in vacuo to yield the uncyclised product as an oil (0.40 g). LRMS (APCI): m/z 320 (MH⁺ of uncyclised product), 302 (MH⁺ less water), 284 (MH⁺ of cyclised product). Potassium hydroxide (0.75 g, 1.33 mmol) was added to a solution of the uncyclised product (0.40 g, 1.23 mmol) in isopropyl alcohol (10 mL) and water (0.4 mL) and stirred at room temperature for 16 hours. The reaction mixture was concentrated in vacuo and partitioned between dichloromethane (30 mL) and water (30 mL). The layers were separated and the aqueous layer re-extracted with dichloromethane (2×20 mL). The combined organics were washed with water (30 mL), dried over anhydrous magnesium sulphate, filtered and concentrated in vacuo to yield the title compound as an oil (0.34 g, 1.19 mmol, 94%). ¹H NMR (CDCl₃, 400 MHz) δ: 0.95 (t, 3H), 1.60-1.70 (m, 2H), 3.30-3.40 (m, 2H), 3.40-3.55 (m, 2H), 3.95 (s, 3H), 4.35 (bd, 1H), 4.42 (bd, 1H), 4.78 (dd, 1H), 6.85 (dd, 1H), 7.00 (s, 1H), 7.38 (dd, 1H). LRMS (APCI): m/z 284 (MH⁺).

### Example 53

### 6-(4-Chloro-3-methoxy-phenyl)-4-propyl-morpholine

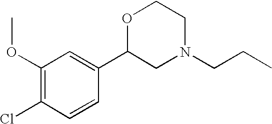

Borane-tetrahydrofuran complex (1M in THF) (3.5 mL, 3.5 mmol) was added dropwise to a solution of the product from example 52 (0.33 g, 1.16 mmol) in dry THF (3 mL) under an atmosphere of nitrogen. The reaction mixture was refluxed for 2.5 hours then cooled and quenched by addition of methanol (1 mL). The reaction mixture was concentrated in vacuo and the residue suspended in 4N HCl (aq., 8 mL) and refluxed for 2 hours. The reaction mixture was cooled and extracted with dichloromethane (2×10 mL). The organic layers were discarded. The aqueous layer was rendered basic (pH 9-10) by addition of solid potassium carbonate before re-extracting with dichloromethane (2×15 mL). The dichloromethane extracts were washed with water (10 mL), dried over anhydrous magnesium sulphate, filtered and concentrated in vacuo to give the title compound as an oil (0.31 g, 1.15 mmol, 99%). ¹H NMR (CDCl₃, 400 MHz) δ: 0.95 (t, 3H), 1.45-1.60 (m, 2H), 2.00 (t, 1H), 2.20 (t, 1H), 2.35 (t, 2H), 2.80 (d, 1H), 2.90 (d, 1H), 3.80 (t, 1H), 3.90 (s, 3H), 4.03 (dd, 1H), 4.55 (d, 1H), 6.85 (dd, 1H), 7.00 (s, 1H), 7.30 (dd, 1H). LRMS (APCI): m/z 270 (MH⁺).

### Example 54

### 2-Chloro-(4-propyl-morpholin-2-yl)-phenol

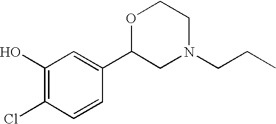

Prepared following the same method as for example 7b (although refluxing was continued for 2.5 hours rather than 1 hour) with the product from example 53 (0.28 g, 1.02 mmol). Purification by column chromatography on silica was not required. The title compound was yielded as a pale brown gum (0.21 g, 0.82 mmol, 81%). ¹H NMR (CDCl₃, 400 MHz) δ: 0.93 (t, 3H), 1.55 (q, 2H), 2.0 (t, 1H), 2.20 (dt, 1H), 2.30-2.40 (m, 2H), 2.80 (bd, 1H), 2.90 (bd, 1H), 3.80 (dt, 1H), 4.0 (dd, 1H), 4.30 (d, 1H), 6.87 (dt, 1H), 7.02 (fd, 1H), 7.25 (s, 1H). LRMS (APCI): m/z 256 (MH⁺). Analysis found C, 60.71%; H, 7.10%; N, 5.45%. C₁₃H₁₈NO₂Cl requires C, 61.05%; H, 7.09%; N, 5.48%.

### Example 55

### Methyl (2S)-2-(propionylamino)propanoate

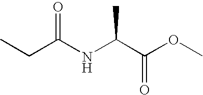

L-Alanine methyl ester hydrochloride salt (14 g, 0.1 mol) was dissolved in dichloromethane (150 mL) and treated with triethylamine (30.45 g, 0.3 mmol). The solution was stirred and propionyl chloride added dropwise. After stirring overnight the mixture was quenched by addition of 1M hydrochloric acid (200 mL) and the organic layer separated. The aqueous layer was re-extracted with dichloromethane (3×200 mL) and the combined organic layers were dried with magnesium sulfate, filtered and evaporated to a to a clear oil (16.0 g, quant.).

¹H NMR (DMSO-d6, 400 MHz) δ: 0.95 (t, 3H), 1.25 (d, 3H), 2.1 (q, 2H), 3.6 (s, 3H), 4.2 (quin, 1H), 8.2 (bd, 1H). LRMS (ESI+) m/z 160 (MH⁺)

### Example 56

### tert-butyl (1S)-2-hydroxy-1-methylethyl(propyl)carbamate

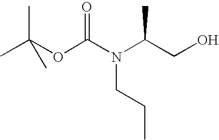

The product from example 55 was dissolved in tetrahydrofuran (200 mL) and borane-tetrahydrofuran complex (300 mL, 0.3 mol) was added to the stirred solution at room temperature. The mixture was then heated at reflux overnight. After to cooling to room temperature, the reaction was quenched by the cautious addition of 6M hydrochloric acid (100 mL) and then heated to reflux for 6 hours. The reaction mixture was allowed to cool to room temperature overnight, and then evaporated to dryness (11.77 g). The crude mixture gave m/z 118 consistent with the desired aminoalcohol intermediate. The crude mixture was then dissolved in methanol (50 mL) and water (400 mL) before the addition of potassium hydroxide (28.22 g, 0.5 mol). Di-tert-butyl dicarbonate (32.87 g 0.15 mol) was added to the mixture and stirring continued over 3 days. The reaction mixture was partitioned between DCM (500 mL) and water (100 mL), the organic layer separated and the aqueous layer re-extracted with DCM twice more. The combined organic fractions were dried with magnesium sulfate, filtered and evaporated to a crude. Purification by flash chromatography on SiO₂ eluting with dichloromethane:methanol:880 NH₃ (97:3:0.3), afforded the desired product as a clear oil 4.5 g (21%) together with a further 10 g of partially purified material.

¹H NMR (DMSO-d6, 400 MHz) δ: 0.8 (t, 3H), 1.05 (bs, 3H), 1.4 (m, 11H), 2.95 (bs, 2H), 3.35 (bm, 3H), 4.6 (bs, 1H) LRMS (ESI+) m/z 240 (MNa⁺)

### Example 57

### (2S)-2-(propylamino)propan-1-ol hydrochloride

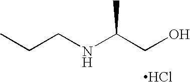

The pure material from example 56 (4.2 g, 0.021 mol) was dissolved in dioxan (10 mL) and treated with 4M HCl in dioxan (30 mL). The mixture was stirred at room temperature for 16 hours and then evaporated to a white solid (2.74 g, 92%)

¹H NMR (DMSO-d6, 400 MHz) δ: 0.9 (t, 3H), 1.15 (d, 3H), 1.6 (m, 2H), 2.8 (m, 2H), 3.15 (m, 1H), 3.5 (bm, 1H), 3.6 (m, 1H), 5.4 (bs, 1H), 8.8 (bd, 2H). LRMS (APCI+) 118 (MH⁺)

### Example 58

### (5S)-2-(3-methoxyphenyl)-5-methyl-4-propylmorpholin-2-ol

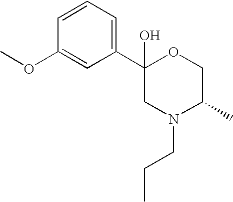

The product from example 57 (1.0 g, 6.6 mmol) was dissolved in toluene (10 mL) and treated with triethylamine (1.38 g, 14 mmol) before the addition of 2-bromo-3′-methoxyacetophenone (1.5 g, 6.6 mmol). The mixture was heated to 65° C. and stirred over 3 days. After cooling to room temperature the mixture was partitioned between brine and ethyl acetate, the organic layer separated, dried with magnesium sulfate, filtered and evaporated. The residue was purified by flash chromatography on SiO₂ eluting with ethyl acetate, to afford the desired morpholinol compound as a mixture of stereoisomers as a pale yellow oil (1.0 g 58%).

¹H NMR (DMSO-d6, 400 MHz) δ: 0.8 (m, 3H), 0.95 (d, 3H), 1.35 (m, 2H), 2.1 (m, 2H), 2.4 (bm, 1H), 2.6 (m, 1H), 2.75 (m, 1H), 3.5 (d, 1H), 3.75 (m, 4H), 6.0 (s, 0.75H), 6.1 (s, 0.25H), 6.85 (d, 1H), 7.05 (m, 2H), 7.25 (t, 1H). LRMS (ESI+) m/z 248 (M-H2O), 266 (MH⁺), 288 (MNa+)

### Example 59

### (5S)-2-(3-methoxyphenyl)-5-methyl-4-propylmorpholine

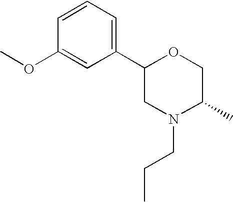

The product from example 58 (770 mg, 2.9 mmol) was dissolved in dichloromethane (20 mL) and cooled to −78° C. under a nitrogen atmosphere. Triethylsilane (3.7 mL, 23 mmol) was added to the stirred mixture followed by trimethylsilyltriflate (1.1 mL, 5.8 mmol). Stirring was continued overnight and the reaction mixture allowed to reach room temperature. The reaction was quenched by the addition of saturated aqueous sodium bicarbonate solution and extracted with dichloromethane (three times). The combined organic layers were dried with magnesium sulfate, filtered and evaporated. The crude product was purified by flash chromatography on SiO₂ dichloromethane:methanol:880 ammonia (97:3:0.3), to yield the desired morpholine compound (600 mg, 83%)

¹H NMR (CDCl₃, 400 MHz) δ: 0.95 (m, 3H), 1.1 (b, d, 3H), 1.6 (bm, 2H), 2.2-3.1 (5H), 3.5 (bm, 1H), 4.85 (m, 4H), 4.6 (b, 1H), 6.8 (d, 1H), 6.95 (m, 2H), 7.25 (m, 1+CHCl₃)

LRMS (APCI+) m/z 250 (MH⁺)

Analysis found C, 71.53%; H, 9.21%; N, 5.55%. C₁₅H₂₃NO₂.0.15H₂O requires C, 71.48%; H, 9.32%; N, 5.56%.

### Example 60

### 3-[(5S)-5-methyl-4-propylmorpholin-2-yl]phenol

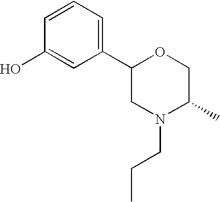

The material from example 59 (400 mg, 1.6 mmol) was dissolved in 48% aqueous hydrobromic acid (8 mL) and the mixture heated to 80° C. overnight. After cooling to room temperature, the mixture was quenched by the addition of saturated aqueous sodium bicarbonate, and the mixture extracted with dichloromethane (three times). The combined organic layers were dried with magnesium sulfate, filtered and evaporated to give the products as a white solid (285 mg, 76%)

¹H NMR (CDCl₃, 400 MHz) δ: 0.9 (m, 3H), 1.1+1.2 (2×d, 3H), 1.5 (m, 2H), 2.3 (m, 2H), 2.5 (bm, 1H), 2.8 (bm, 1H), 3.1 (d, 1H), 3.5 (bm, 1H), 3.85 (bm, 1H), 4.6 (d, 1H), 6.8 (m, 2H), 6.95 (m, 1H), 7.2 (t, 1H)

LRMS (APCI+), 236 (MH⁺)

Analysis found C, 70.61%; H, 9.00%; N, 5.86%. C₁₄H₂₁NO₂.0.1H₂0 requires C, 70.91%; H, 9.01%; N, 5.91%.

This mixture of diastereoisomers was separated on a Chiralcel OJ-H (250*21.2 mm) HPLC column. Mobile phase 100% MeOH, flow rate 15 ml/min.

Sample preparation 200 mg dissolved in 4 ml MeOH, 250 μL injection.

Two major peaks were obtained, with retention times 5.822 min (example 60A, 57 mg 28%) and 7.939 min (example 60B, 12 mg, 6%)

Example 60A: ¹H NMR (CDCl₃. 400 MHz) δ: 0.85 (t, 3H), 1.05 (d, 3H), 1.5 (m, 2H+H₂0), 2.2 (m, 2H), 2.4 (m, 1H), 2.8 (m, 1H), 3.0 (d, 1H), 3.4 (t, 1H), 3.9 (dd, 1H), 4.55 (d, 1H), 5.6 (bs, 1H), 6.75 (d, 1H), 6.85 (s, 1H), 6.95 (d, 1H), 7.2 (t, 1H)

HRMS m/z 236.1643 (MH⁺)

Example 60B: ¹H NMR (CDCl₃, 400 MHz) δ: 0.95 (t, 3H), 1.15 (d, 3H), 1.55 (m, 2H), 2.4 (m, 2H), 2.55 (t, 1H), 2.65 (dd, 1H), 2.95 (bm, 1H), 3.8 (d, 1H), 3.95 (d, 1H), 4.55 (dd, 1H), 6.75 (d, 1H), 6.85 (s, 1H), 6.95 (d, 1H), 7.2 (t, 1H)

HRMS m/z 236.1643 (MH⁺)

### Example 61

### (S)-2-propylamino-propan-1-ol hydrochloride

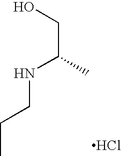

To (S)-(+)-2-amino-1-propanol (19.6 g, 0.26 mol) dissolved in dichloromethane (500 ml) was added propionaldehyde (20.9 ml, 0.28 mol) followed by pre-dried powdered 4A molecular sieves (40 g) and the mixture stirred at room temperature overnight. The mixture was filtered through a pad of celite, the pad washed with dichloromethane, and solvent evaporated to give a clear oil. This oil was dissolved in methanol (200 ml) and NaBH₄ was added portionwise over 15 minutes. The mixture was stirred at room temperature overnight, then quenched by cautious addition of 2M HCl_{(aq)} (200 ml), basified by addition of 2M NaOH (200 ml) and methanol removed by evaporation. Di-tert-butyldicarbonate (115 g, 0.52 mol) was added followed by 1,4-dioxan (200 ml) and the mixture stirred at room temperature overnight. 1,4-dioxan was removed by evaporation giving a clear oil. To this oil was added 4M HCl in 1,4-dioxan (200 ml) and the mixture stirred at room temperature overnight. The solvent was removed by evaporation to give a white solid (24 g).

¹H NMR (DMSO, 400 MHz) δ: 0.95 (t, 3H), 1.2 (d, 3H), 1.6 (m, 2H), 2.8 (m, 2H), 3.15 (m, 1H), 3.5 (bm, 1H), 3.6 (m, 1H), 5.4 (b, 1H), 8.6-8.9 (bd, 2H)

LRMS (APCI+), 118 (MH⁺)

### Example 62

### (5S)-4-propyl-5-methylmorpholin-2-one

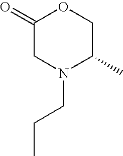

The material from example 61 (4 g, 26 mmol) was dissolved in benzene, followed by the addition of N-ethyldiisopropylamine (9.07 ml, 52 mmol) and methyl bromoacetate (2.4 ml, 26 mmol). The mixture was heated to reflux with azeotropic removal of water overnight. The solvent was removed by evaporation, the crude material dissolved in methanol, pre-absorbed onto SiO₂ and flash chromatographed on SiO₂ eluting with 40% EtOAc/Pentane to afford the title morpholinone as a clear oil (1.78 g).

¹H NMR (CDCl₃, 400 MHz): 0.9 (t, 3H), 1.1 (d, 3H), 1.5 (m, 2H), 2.25 (m, 1H), 2.6 (m, 1H), 2.8 (m, 1H), 3.2 (d, 1H), 3.6 (d, 1H), 4.05 (dd, 1H), 4.3 (dd, 1H)

t.l.c. Rf=0.18 (50% EtOAc/Pentane, UV visualisation)

### Example 63

### (5S)-2-[6-(2,5-dimethyl-1H-pyrrol-1-yl)pyridin-3-yl]-4-propyl-5-methylmorpholin-2-ol

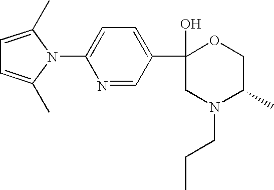

5-bromo-2-(2,5-dimethyl-pyrrol-1-yl)-pyridine (1.5 g 5.9 mmol) was azeotroped with toluene and dissolved in THF (20 ml). This mixture was cooled to −78 C and t-butyllithium (1.7M in pentane, 7 ml, 11.9 mmol) was added maintaining the temperature below −70° C. The material from example 62 was dissolved in THF (20 ml) and added to the mixture immediately on completion of the t-butyllithium addition. The mixture was allowed to stir at −78° C. for 30 minutes at which time NH₄Cl (10% aq, 150 ml) was added and the mixture extracted into EtOAc (200 ml), dried with magnesium sulphate, filtered and evaporated. Flash chromatography on SiO₂ eluting with a stepped gradient from 25% EtOAc/pentane to 50% EtOAc/pentane gave the title compound as mixture of diastereoisomers in approximately 3.5:1 ratio as a yellow oil (480 mg).

¹H NMR (CDCl₃, 400 MHz) (diastereomers) δ: 0.95 (m, 3H), 1.1, 1.2 (2×d, 3H) 1.5 (m, 2H), 2.15 (s, 6H), 2.4 (m, 1H), 2.5 (d, 1H), 2.6 (m, 1H), 2.75 (m, 1H), 3.85-3.95 (m, 1H), 3.6, 3.75, 4.4 (3×m, 2H), 5.15 (bs, 1H), 5.9 (s, 2H), 7.2 (d, 1H), 8.05 (dd, 1H), 8.8 (s, 1H)

LRMS (ES+), 330 (MH⁺), 352 (MNa+)

LRMS (ES−), 328 (M-H)

### Example 64

### (2S)-2-[{(2RS)-2-[6-(2,5-dimethyl-1H-pyrrol-1-yl)pyridin-3-yl]-2-hydroxyethyl}propyl)amino]propan-1-ol

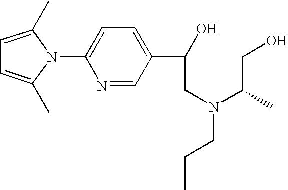

(5S)-2-[6-(2,5-dimethyl-1H-pyrrol-1-yl)pyridin-3-yl]-4-propyl-5-methylmorpholin-2-ol (480 mg, 1.45 mmol) was dissolved in ethanol (5 mL) and water (2 mL) and treated with sodium borohydride (220 mg, 5.8 mmol). The reaction mixture was left stirring overnight at room temperature before being quenched by the addition of saturated aqueous NH₄Cl (50 mL) and extracted with ethyl acetate (2×100 mL). The organic extracts were combined, dried with MgSO₄ and evaporated to give 400 mg of a fluffy white solid which was used without further purification

¹H NMR (CDCl₃, 400 MHz) diastereomers δ: 0.8-1.1 (m, 6H), 1.15, 1.35 (2×d, 3H), 1.6-2.0 (m, 2H) 2.1 (s, 6H), 2.5-4.05 (m, 7H), 4.8-5.2 (m, 1H), 5.9 (s, 2H), 7.2 (m, 1H), 7.8-8.1 (m, 1H), 8.55 (m, 1H).

LRMS (ES+), 332 (MH⁺)

### Example 65

### (2S)-2-[[(2RS)-2-(6-aminopyridin-3-yl)-2-hydroxyethyl](propyl)amino]propan-1-ol

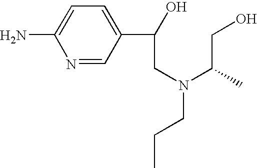

(2S2-[[(2RS)-2-(6-aminopyridin-3-yl)-2-hydroxyethyl](propyl)amino]propan-1-ol (400 mg, 1.2 mmol) was dissolved in EtOH (5 mL), hydroxylamine hydrochloride (419 mg, 6 mmol) was added and the mixture heated to 80° C. overnight. The solvent was removed under vacuum and the residue purified by flash chromatography on SiO₂ eluting with dichloromethane/methanol/880 ammonia (95:5:0.5 increasing polarity to 93:7:1) to afford the title compounds as a mixture of diastereoisomers (300 mg, 98%)

¹H NMR (CDCl₃, 400 MHz) (2 diastereomers) δ: 0.82-0.97 (6H, m), 2.40-2.77 (2H, m), 3.27-3.51 (2H, m), 4.51 (1H, m), 6.58 (1H, m), 7.49 (1H, m), 7.86 (1H, m)

LRMS (APCI+), 254 (MH⁺)

### Examples 66 and 67

### 5-[(2S,5S)-5-methyl-4-propylmorpholin-2-yl]pyridin-2-amine and 5-[(2R,5S)-5-methyl-4-propylmorpholin-2-yl]pyridin-2-amine

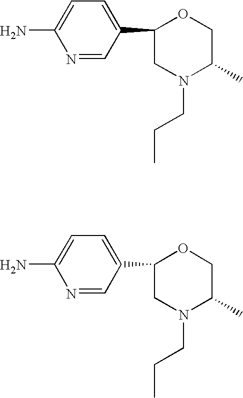

The “diol” from example 65 (300 mg, 1.2 mmol) was dissolved in dichloromethane (3 mL), and concentrated sulphuric acid (3 mL) was added. The mixture was stirred at room temperature for 3 hours. The reaction was cooled to 0° C., quenched by the cautious addition of 6M sodium hydroxide solution and then extracted with dichloromethane (4×50 mL). The combined extracts were dried (MgSO₄) and evaporated to a brown gummy solid. Purification by flash chromatography on SiO₂ eluting with 10% methanol in ethyl acetate afforded 5 mg of material enriched in the less polar diastereomer (ca. 80% d.e.), 12 mg of material enriched in the less polar diastereomer (ca. 80% d.e.) and 150 mg of material ca. 1:1 mixture of diastereoisomers (total yield 167 mg, 59%). The latter 1:1 mixture was subjected to purification by HPLC using a Chiralpak OD-H column (250×21.2 mm), eluting with methanol/ethanol (1:1).

The faster eluting diastereoisomer (retention time 6.1 min) was obtained in >99% d.e (60 mg, 21%). ¹H NMR (CDCl₃, 400 MHz) 0.88 (3H, t), 1.01 (3H, d), 1.26 (3H, t), 1.37-1.58 (2H, m), 2.18-2.28 (2H, m), 2.36-2.47 (1H, m), 2.69-2.77 (1H, m), 2.90 (1H, m), 3.38 (1H, m), 3.72 (2H, d), 3.82 (1H, m), 4.40 (2H, brs), 4.45 (1H, dd), 6.48 (1H, d), 7.45 (1H, dd), 8.04 (1H, d)

LRMS (ES⁺): m/z 236 (MH⁺)

[α]_{D}²⁵ 46.28 (c 0.13, MeOH)

The slower eluting diastereoisomer (retention time 10.5 min) was obtained in >99% d.e. (62 mg, 22%). ¹H NMR (CDCl₃, 400 MHz) 0.93 (3H, t), 1.11 (3H, d), 1.49 (2H, m), 2.38 (2H, m), 2.50-2.56 (2H, m), 2.89 (1H, m), 3.75 (1H, m), 3.89 (1H, m), 4.40 (2H, brs), 4.46 (1H, m), 6.50 (1H, d), 7.50 (1H, dd), 8.07 (1H, d)

LRMS (ES⁺): m/z 236 (MH⁺)

[α]_{D}²⁵ 22.58 (c 0.13, MeOH)

The activity of a compound in the treatment of pain may be measured according to the following test protocols.

Mono-Iodoacetate (MIA)-Induced OA model

Male Sprague-Dawley rats (125-175 g) are anesthetized with a 2% isofluorane-O₂ mixture and given a unilateral intraarticular injection of monosodium iodoacetate (MIA; Sigma, Poole, UK) through the infrapatella ligament of the right knee as described by Dunham et al (J Exp Pathol, 74: 283-289, 1993). MIA was dissolved in 0.9% sterile saline and administered in a volume of 25 μl using a 26 gauge, 0.5 inch needle.

The effect of joint damage on the weight distribution through the ipsilateral (arthritic) and contralateral (untreated) knee was assessed using an incapacitance tester (Linton Instrumentation). Briefly, the incapacitance tester measures weight distribution on the two hind paws. The force exerted by each hind limb is measured in grams.

The weight-bearing (WB) deficit is defined as the difference between the amounts of weight measured in the contralateral paw and ipsilateral paw. The WB is measured at various time points after the administration of the test compounds or vehicle.

### Assessment of Static and Dynamic Allodynia

### Static Allodynia

Animals were habituated to wire bottom test cages prior to the assessment of allodynia. Static allodynia was evaluated by application of von Frey hairs (Stoelting, Wood Dale, Ill., USA.) in ascending order of force (0.6, 1, 1.4, 2, 4, 6, 8, 10, 15 and 26 grams) to the plantar surface of hind paws. Each von Frey hair was applied to the paw for a maximum of 6 sec, or until a withdrawal response occurred. Once a withdrawal response to a von Frey hair was established, the paw was re-tested, starting with the filament below the one that produced a withdrawal, and subsequently with the remaining filaments in descending force sequence until no withdrawal occurred. The highest force of 26 g lifted the paw as well as eliciting a response, thus represented the cut off point. Each animal had both hind paws tested in this manner. The lowest amount of force required to elicit a response was recorded as paw withdrawal threshold (PWT) in grams. Static allodynia was defined as present i animals responded to a stimulus of, or less than, 4 g, which is innocuous in naive rats (Field M J, Bramwell S, Hughes J, Singh L. Detection of static and dynamic components of mechanical allodynia in rat models of neuropathic pain: are they signalled by distinct primary sensory neurones? Pain, 1999; 83:303-11).

### Dynamic Allodynia

Dynamic allodynia was assessed by lightly stroking the plantar surface of the hind paw with a cotton bud. To avoid recording general motor activity, care was taken to perform this procedure in fully habituated rats that were not active. At least two measurements were taken at each time point, the mean of which represented the paw withdrawal latency (PWL). If no reaction was exhibited within 15 sec the procedure was terminated and animals were assigned this withdrawal time. A pain withdrawal response was often accompanied with repeated flinching or licking of the paw. Dynamic allodynia was considered to be present if animals responded to the cotton stimulus within 8 sec of commencing stroking (Field et al, 1999).

### Hotplate

Experimental procedure: Male Sprague-Dawley rats (125-250 g) are placed on a hot plate (Ugo Basile, Italy) maintained at 55±5° C. The time between placement of the animal on the hot plate and occurrence of either licking of fore or hind paw, shaking or jumping off the surface is measured. Baseline measurements will be made and animals reassessed following drug administration. The cut off time for hot plate latencies is set at 20 seconds to prevent tissue damage.

### Chronic Constriction Injury (CCl) Rat Model of Neuropathic Pain

The CCl of sciatic nerve was performed as previously described by Bennett and Xie (Bennett G J, Xie Y K. A peripheral mononeuropathy in rat that produces disorders of pain sensation like those seen in man. Pain:33:87-107, 1988). Animals were anaesthetized with a 2% isofluorane/O₂ mixture. The right hind thigh was shaved and swabbed with 1% iodine. Animals were then transferred to a homeothermic blanket for the duration of the procedure and anesthesia maintained during surgery via a nose cone. The skin was cut along the line of the thighbone. The common sciatic nerve was exposed at the middle of the thigh by blunt dissection through biceps femoris. About 7 mm of nerve was freed proximal to the sciatic trifurcation, by inserting forceps under the nerve and the nerve gently lifted out of the thigh. Suture was pulled under the nerve using forceps and tied in a simple knot until slight resistance was felt and then double knotted. The procedure was repeated until 4 ligatures (4-0 silk) were tied loosely around the nerve with approx 1 mm spacing. The incision was closed in layers and the wound treated with topical antibiotics.

### In Vitro Pharmacology Studies

5-[(2R,5S)-5-methyl-4-propylmorpholin-2-yl]pyridin-2-amine (the compound of Example 67) is a selective D₃ dopamine receptor agonist that inhibits forskolin-stimulated cAMP accumulation in CHO cells, which are stably expressing the recombinant human D₃ dopamine receptor, with an EC₅₀ of 21 nM and behaves as a full agonist (% Eₘₐₓ=95). Furthermore, the compound of Example 67 displays >470- and 180-fold functional selectivity over D₂ and D₄ dopamine receptors, respectively. In addition, the compound of Example 67 shows a >46- and 19-fold binding selectivity over D₂ and D₄ dopamine receptors, respectively (Table 1).

TABLE 1
In Vitro Pharmacologic Activities of the compound of Example 67

| Activity | Endpoint |
|---|---|
| Functional inhibition of | Geometric mean EC₅₀ (95% CI) (n) |
| forskolin-stimulated | |
| cAMP accumulation in | |
| human recombinant cell | |
| lines | |
| D₃ | EC₅₀ = 21 nM (18-30) (n = 18) |
| | % Eₘₐₓ = 95 (89-103) |
| D₂ | EC₅₀ = >10 μM (n = 4) |
| | % Response @ 10 μM = 36 (19-56) |
| D₄ | EC₅₀ = 3.9 μM(1.6-5.5) (n = 2) |
| | % Eₘₐₓ = 92 (87-96) |
| In vitro binding (membrane | |
| preparation), human | |
| recombinant receptors | |
| D₃ | Kᵢ = 215 nM (157-293) (n = 9) |
| D₁ | IC₅₀ = >10 μM (n = 2) |
| D₂ | IC₅₀ = >10 μM (n = 2) |
| D₄ | Kᵢ = 4165 nM (3670-4727) (n = 4) |
| D₅ | IC₅₀ = >10 μM (n = 2) |

n = Number of separate experiments

%. Response=Response expressed as a percentage of the effect to pramipexole

### In Vivo Pharmacology Studies

### Models of Nociceptive and Neuropathic Pain

The compound of Example 67 has been evaluated using in vivo models to determine the pain state for which D3 agonism will be predicted to be effective.

### Effect of the Compound of Example 67 on Monosodium Iodoacetate-Induced Pain (Mia)

Rats subjected to MIA injection into the knee joint have a progressive degradation of cartilage due to chondrocyte death, which results in behavioral signs of allodynia. The effect of MIA allodynia varies over time, but is most pronounced and consistent between days 12 to 35 following MIA injection. These signs include lowered withdrawal thresholds to mechanical stimulation (static allodynia, approximately 2 g compared with approximately 12 g in normal rats). Data with tramadol and oxycodone indicate that this measure is predictive of the efficacy of compounds for the treatment of nociceptive pain. The compound of Example 67 dosed orally at 0.01, 0.03, and 0.1 mg/kg demonstrated a dose response in reversal of the allodynia, with a full reversal at 0.1 mg/kg similar to the positive control of the opioid tramadol (100 mg/kg). Following these oral doses of the compound of Example 67, the free plasma concentration achieved was 1.85, 5.70, and 10.77 nM, respectively. Furthermore, the effect of 0.1 mg/kg of the compound of Example 67 was abolished following oral dosing at 3 mg/kg of the potent D₃ receptor antagonist quinoline-4-carboxylic acid (4-[2-(6-cyano-3,4-dihydro-1H-isoquinolin-2-yl)ethyl]cyclohexyl)-amide (representing 16.9 nM free plasma antagonist concentration). Using an alternative endpoint of weight-bearing deficit following MIA injection, a single dose of 0.1 mg/kg of the compound of Example 67 also demonstrated efficacy similar to optimal opioid treatment. Together, these data confirm the therapeutic potential of DS agonist activity in the treatment of nociceptive pain states such as OA.

### Effect of the Compound of Example 67 in Postsurgical Pain

The plantar surgery model is a model of postsurgical inflammatory and nociceptive pain. This can be assessed using a measure of static (see MIA Section above) and dynamic allodynia (reduced latency to withdrawal from brush stimuli; approximately 2 seconds compared with >15 seconds in normal rats) to investigate paw withdrawal latency. In this model, an incision is made in the plantaris muscle and static and dynamic allodynia endpoints are measured. In this model, 5-[(2R,5S)-5-methyl-4-propylmorpholin-2-yl]pyridin-2-amine induced a dose-dependent reversal of surgery-induced static and dynamic allodynia, with a maximal effect following an oral dose of 0.1 mg/kg. Based on the known PK profile, the peak free-plasma concentration at this dose is approximately 10 nM (see MIA Section above). This response was similar in magnitude to the positive control of gabapentin (100 mg/kg PO).

### Effect of the Compound of Example 67 in Capsaicin-Induced Hyperalgesia

The capsaicin model of hyperalgesia measures a static and dynamic allodynia endpoint following sensitization by intradermal injection of 30 μg of capsaicin into the foot of the rat. Data demonstrated a dose response following oral pre-dosing of 0.01, 0.1, and 1 mg/kg of the compound of Example 67, followed by capsaicin injection 1 hour after dosing. The optimal effect observed for the compound of Example 67 was following the 1 mg/kg dose of the compound and this was equivalent in magnitude to the positive control of 30 mg/kg of pregabalin.

### Effect of the Compound of Example 67 in Hotplate Model

The hotplate model is an additional nociceptive pain model that measures paw withdrawal latency from a hotplate. In this model, the hotplate is heated to 52° C. and the behavior of the rat is monitored at baseline and following drug administration. The compound of Example 67 dosed orally at 0.01, 0.03. 0.1 and 1.0 mg/kg demonstrated no efficacy in this model, in contrast to the positive control of SC-dosed morphine at 3 mg/kg. These data indicate a lack of efficacy against normal thermal thresholds, which is in contrast to the MIA and plantar surgery induced nociceptive pain models. A key difference that may explain this observation is the use of sensitized/injured animals used in the other models, as opposed to naive animals used in the hotplate test.

### Effect of the Compound of Example 67 on Chronic Constriction Injury

Rats subjected to chronic constriction injury of the sciatic nerve have behavioral signs of allodynia from approximately 14 days after surgery. These signs include allodynia represented by lowered paw withdrawal thresholds following static and dynamic stimulation. The compound of Example 67 demonstrated no effects in this model following oral dosing at 0.1 mg/kg, translating to a predicted free-plasma exposure of 10 nM (see (MIA) Section above).

TABLE 2
In Vitro Pain Pharmacology Endpoints
for the compound of Example 67

| | Minimum Effective Dose |
|---|---|
| Functional Model | (measured mean free Cₘₐₓfor MED) |
| MIA-induced static allodynia (rats) | 0.01 mg/kg oral (1.85 nM) |
| MIA-induced weight deficit (rats) | 0.1 mg/kg oral single-dose study |
| Plantar surgery-induced static | |
| and dynamic allodynia (rats) | |
| Capsaicin-induced hyperalgesia | 0.01 mg/kg oral |
| Hotplate paw withdrawal latency | No effect up to 1.0 mg/kg oral |
| (rats) | |
| CCI-induced static and dynamic | No effect up to 0.1 mg/kg oral |
| allodynia (rats) | |

1. A method of treating pain comprising the administration to a mammal of an effective amount of a compound of formula (I), (Ia) or (Ib) 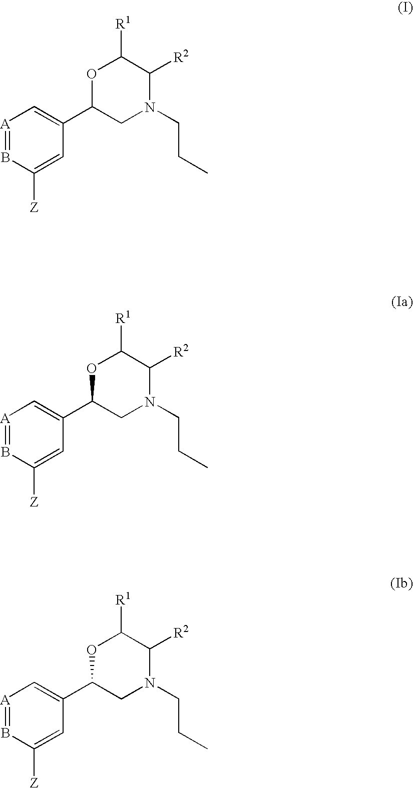
wherein:
A is C—X or N;
B is C—Y or N;
R¹ is H or (C₁-C₆)alkyl;
R² is H or (C₁-C₆)alkyl;
X is H, HO, C(O)NH₂ or NH₂;
Y is H, HO, NH₂, Br, Cl or F;
Z is H, HO, F, CONH₂ or CN;
or a pharmaceutically acceptable salt.

2. A method according to claim 1 in which said pain is selected from the group consisting of chronic pain and nociceptive pain.

3. A method according to claim 1 wherein A is C—X or N and B is C—Y.

4. A method according to claim 1 wherein R¹ is H, methyl or ethyl.

5. A method according to claim 1 wherein R² is H or methyl.

6. A method according to claim 1 wherein X is H, OH or NH₂.

7. A method according to claim 1 wherein Y is H, NH₂, Cl or F.

8. A method according to claim 1 wherein Z is H, OH or F.

9. A method according to claim 1 wherein the compound is selected from the group consisting of:
(R)-(−)-3-Propylmorpholin-2-yl)phenol;
(S)-(+)-3-(4-Propylmorpholin-2-yl)phenol;
(R)-(−)-3-(4-Propylmorpholin-2-yl)phenol hydrochloride;
(R)-5-(4-Propylmorpholin-2-yl)benzene-1,3-diol;
(S)-5-Propylmorpholin-2-yl)benzene-1,3-diol;
(R)-(+)-2-Fluoro-5-(4-propylmorpholin-2-yl)phenol;
(S)-(−)-2-Fluoro-5-(4-propylmorpholin-2-yl)phenol;
5-(4-Propylmorpholin-2-yl)pyridin-2-ylamine;
2-Chloro-54-propyl-morpholin-2-yl)phenol;
5-[(2S,5S)-5-methyl-4-propylmorpholin-2-yl]pyridin-2-amine; and
5-[(2R,5S)-5-methyl-4-propylmorpholin-2-yl]pyridin-2-amine;
or a pharmaceutically acceptable salt thereof.

10. A method according to claim 1 wherein the compound is 5-[(2R,5S)-5-methyl-4-propylmorpholin-2-yl]pyridin-2-amine, or a pharmaceutically acceptable salt thereof.

11. A method according to claim 1 in which said pain is chronic pain.

12. A method according to claim 1 wherein the pain is chronic nociceptive pain.

13. A method according to claim 1 wherein the pain is nociceptive pain.

14. A method according to claim 1 wherein the pain is associated with osteoarthritis.

15. A method according to claim 1 wherein the pain is post-surgical pain.

16. A method according to claim 1 wherein the pain is neuropathic pain.

17. A method according to claim 1 wherein the pain is visceral pain.

18. A method according to claim 1 wherein the pain is inflammatory pain.

19. A method according to claim 1 in which said compound is selected from the group consisting A and B below, or a pharmaceutically acceptable salt thereof:
A. (R)-(−)-3-(4-Propylmorpholin-2-yl)phenol;
B. (S)-(+)-3-(4-Propylmorpholin-2-yl)phenol.
